(19) United States
(12) Patent Application Publication
(10) Pub. No.: US 2008/0318933 A1
(43) Pub. Date: Dec. 25, 2008

(54) 5-Sulfonyl-1-Piperidinyl Substituted Indole Derivatives as 5-Ht6 Receptor Antagonists for the Treatment of Cns Disorders

(75) Inventors: Mahmood Ahmed, Essex (GB); Christopher Norbert Johnson, Essex (GB); Neil Derek Miller, Essex (GB); Peter Henry Milner, Essex (GB); Dean Andrew Rivers, Essex (GB); David R Witty, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 11/576,070

(22) PCT Filed: Oct. 5, 2005
(86) PCT No.: PCT/GB05/03835

§ 371 Date: Mar. 27, 2007

(30) Foreign Application Priority Data
Oct. 7, 2004 (GB) 0422263.4

(51) Int. Cl. A61K 31/55; C07D 401/04; A61K 31/454; A61P 3/00; A61P 25/00; C07D 403/04; A61K 31/403

(52) U.S. Cl. 514/217.08; 546/201; 514/339; 548/465; 514/414; 540/602

(57) ABSTRACT

The present invention relates to novel indole derivatives such as compounds of the formula (I):

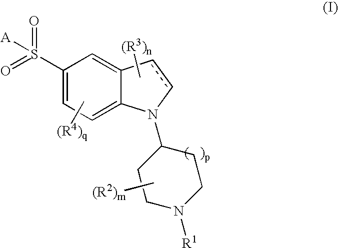

which possess antagonist potency at the 5-HT₆ receptor and the use of such compounds or pharmaceutically acceptable salts or solvates thereof in the treatment of Alzheimer's disease and other CNS disorders.

This invention relates to novel indole derivatives having pharmacological activity, to processes for their preparation, to compositions containing them and to their use in the treatment of CNS and other disorders.

The background to the present invention includes the following publications:
DE19838666 (Mueller, T) describes preparation of indole derivatives by intramolecular reaction of alkynes in the presence of a heterogenous catalyst.
WO 99/33800 (Hoechst) describes a series of indole derivatives as inhibitors of Factor Xa.
WO 99/43654 (Genetics Institute Inc.) describes a series of indole derivatives claimed to be useful as phospholipase inhibitors in the treatment of inflammation.
WO 02/085892 (Wyeth) describes a series of aminobenzazole derivatives as 5-HT₆ ligands which are claimed to be useful for central nervous system disorders.

A structurally novel class of compounds has now been found which possess antagonist potency at the 5-HT₆ receptor. Compounds which possess antagonist potency at the 5-HT₆ receptor are capable of interfering with the physiological effects of 5-HT at the 5-HT₆ receptor and may be antagonists or inverse agonists.

The present invention therefore provides, in a first aspect, a compound of formula (I) or a pharmaceutically acceptable salt thereof:

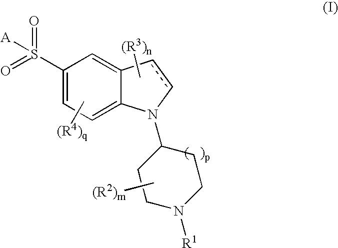

wherein:
R¹ represents hydrogen or C₁₋₆ alkyl optionally substituted by one or more (e.g. 1, 2 or 3) halogen or cyano groups;
R² represents C₁₋₆ alkyl or R² may be linked to R¹ to form a (CH₂)₂, (CH₂)₃ or (CH₂)₄ group;
m represents an integer from zero to 4, such that when m is greater than 1, two R² groups may be linked to form a CH₂, (CH₂)₂, CH₂OCH₂ or (CH₂)₃ group;
p represents an integer from zero to 2; [symbol]
represents a single or a double bond;
R³ represents C₁₋₆ alkyl or ═O;
n represents an integer from zero to 2;
R⁴ represents halogen, cyano, haloC₁₋₆ alkyl, haloC₁₋₆ alkoxy, C₁₋₆ alkyl, C₁₋₆ alkoxy, C₁₋₆ alkanoyl or a group —CONR⁵R⁶;
q represents an integer from zero to 3;
R⁵ and R⁶ independently represent hydrogen or C₁₋₆ alkyl or together with the nitrogen atom to which they are attached form a nitrogen containing heterocyclyl or nitrogen containing heteroaryl group;
A represents an -aryl, -heteroaryl, -aryl-aryl, -aryl-heteroaryl, -heteroaryl-aryl or -heteroaryl-heteroaryl group;
wherein said aryl and heteroaryl groups of A may be optionally substituted by one or more (e.g. 1, 2 or 3) substituents which may be the same or different, and which are selected from the group consisting of halogen, hydroxy, cyano, nitro, trifluoromethyl, trifluoromethoxy, C₁₋₆ alkyl, trifluoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₆ alkyl, C₁₋₆ alkylsulfonamido, C₁₋₆ alkylamido, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group CONR⁹R¹⁰ or SO₂NR⁹R¹⁰, wherein R⁹ and R¹⁰ independently represent hydrogen or C₁₋₆ alkyl or R⁹ and R¹⁰ together with the nitrogen atom to which they are attached may form a nitrogen containing heterocyclyl or nitrogen containing heteroaryl group;
or solvates thereof.

As used herein, the term “alkyl” (when used as a group or as part of a group) refers to a straight or branched hydrocarbon chain containing the specified number of carbon atoms. For example, C₁₋₆ alkyl means a straight or branched hydrocarbon chain containing at least 1 and at most 6 carbon atoms. Examples of alkyl include, but are not limited to; methyl (Me), ethyl (Et), n-propyl, i-propyl, n-hexyl and i-hexyl.

As used herein, the term “alkoxy” (when used as a group or as part of a group) refers to an alkyl ether radical, wherein the term “alkyl” is defined above. Examples of alkoxy include, but are not limited to; methoxy, ethoxy, n-propoxy, i-propoxy, n-pentloxy and i-pentoxy.

The term ‘C₃₋₇ cycloalkyl’ as used herein refers to a saturated monocyclic hydrocarbon ring of 3 to 7 carbon atoms. Examples of such groups include cyclopropyl, cyclobutyl, cyclopentyl, cyclohexyl and cycloheptyl.

The term ‘halogen’ is used herein to describe a group selected from fluorine, chlorine, bromine and iodine.

The term ‘haloC₁₋₆ alkyl’ as used herein refers to a C₁₋₆ alkyl group as defined herein wherein at least one hydrogen atom is replaced with a halogen atom. Examples of such groups include fluoroethyl, trifluoromethyl or trifluoroethyl and the like.

The term ‘haloC₁₋₆ alkoxy’ as used herein refers to a C₁₋₆ alkoxy group as herein defined wherein at least one hydrogen atom is replaced with a halogen atom. Examples of such groups include difluoromethoxy or trifluoromethoxy and the like.

The term ‘aryl’ as used herein refers to a C₆₋₁₂ monocyclic or bicyclic hydrocarbon ring wherein at least one ring is aromatic. Examples of such groups include phenyl, naphthyl or tetrahydronaphthalenyl and the like.

The term ‘heteroaryl’ as used herein refers to a 5-6 membered monocyclic aromatic or a fused 8-10 membered bicyclic aromatic ring containing 1 to 4 heteroatoms selected from oxygen, nitrogen and sulphur. Examples of such monocyclic aromatic rings include thienyl, furyl, furazanyl, pyrrolyl, triazolyl, tetrazolyl, imidazolyl, oxazolyl, thiazolyl, oxadiazolyl, isothiazolyl, isoxazolyl, thiadiazolyl, pyranyl, pyrazolyl, pyrimidyl, pyridazinyl, pyrazinyl, pyridyl, triazinyl, tetrazinyl and the like. Examples of such fused aromatic rings include quinolinyl, isoquinolinyl, quinazolinyl, quinoxalinyl, pteridinyl, cinnolinyl, phthalazinyl, naphthyridinyl, indolyl, isoindolyl, azaindolyl, indolizinyl, indazolyl, purinyl, pyrrolopyridinyl, furopyridinyl, benzofuranyl, isobenzofuranyl, benzothienyl, benzoimidazolyl, benzoxazolyl, benzoisoxazolyl, benzothiazolyl, benzoisothiazolyl, benzoxadiazolyl, benzothiadiazolyl and the like.

Heteroaryl groups, as described above, may be linked to the remainder of the molecule via a carbon atom or, when present, a suitable nitrogen atom except where indicated otherwise.

The term “nitrogen containing heteroaryl” is intended to represent any heteroaryl group as defined above which contains a nitrogen atom.

It will be appreciated that wherein the above mentioned aryl or heteroaryl groups have more than one substituent, said substituents may be linked to form a ring.

The term ‘heterocyclyl’ refers to a 4-7 membered monocyclic saturated or partially unsaturated ring containing 1 to 4 heteroatoms selected from oxygen, nitrogen or sulphur; or a fused 8-12 membered bicyclic saturated or partially unsaturated ring system containing 1 to 4 heteroatoms selected from oxygen, nitrogen or sulphur. Examples of such monocyclic rings include pyrrolidinyl, azetidinyl, pyrazolidinyl, oxazolidinyl, piperidinyl, piperazinyl, morpholinyl, thiomorpholinyl, thiazolidinyl, hydantoinyl, valerolactamyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl, diazepanyl, azepanyl and the like. Examples of such bicyclic rings include indolinyl, isoindolinyl, benzopyranyl, quinuclidinyl, 2,3,4,5-tetrahydro-1H-3-benzazepine, tetrahydroisoquinolinyl and the like.

The term ‘nitrogen containing heterocyclyl’ is intended to represent any heterocyclyl group as defined above which contains a nitrogen atom.

In one embodiment there is provided a compound of formula (I), or a pharmaceutically acceptable salt thereof, wherein:

R¹ represents hydrogen or C₁₋₆ alkyl;
m represents 0 or 1;
R² represents C₁₋₃ alkyl or R² may be linked to R¹ to form a (CH₂)₃ group;
n represents 0, 1 or 2;
R³ represents C₁₋₃ alkyl;
p represents 0, 1, or 2;
q represents 0 or 1;
R⁴ represents halogen; and
A represents an optionally substituted phenyl, thiazolyl or pyrazolyl, wherein the optional substituents are selected from the group consisting of halogen, CN, C₁₋₃ alkyl and C₁₋₃ alkoxy;
or solvates thereof.

In certain embodiments, R¹ represents hydrogen or C₁₋₆ alkyl (e.g. methyl, ethyl, n-propyl, i-propyl or 2,2-dimethylpropyl). In one embodiment, R¹ represents hydrogen or methyl.

In one embodiment, m represents 0 or 1, more particularly 0.

In one embodiment, R² represents C₁₋₃ alkyl (e.g. methyl) or R² may be linked to R¹ to form a (CH₂)₃ group.

In one embodiment, n represents 0 or 1, more particularly 0.

In one embodiment, R³ represents C₁₋₃ alkyl (e.g. methyl).

In one embodiment, n represents 2 and R³ represents methyl.

In one embodiment, p represents 0, 1, or 2, more particularly 1.

In one embodiment, q represents 0 or 1, more particularly 0.

In one embodiment, R⁴ represents halogen, more particularly F or Cl.

In one embodiment, A represents an optionally substituted phenyl, thiazolyl or pyrazolyl, more particularly phenyl, wherein the optional substituents are selected from the group consisting of halogen (e.g. F or Cl), CN, C₁₋₃ alkyl (e.g. methyl) and C₁₋₃ alkoxy (e.g. methoxy).

Preferred compounds according to the invention include examples E1-E65 as shown below, or a pharmaceutically acceptable salt or solvate thereof.

The compounds of formula (I) can form acid addition salts thereof. It will be appreciated that for use in medicine the salts of the compounds of formula (I) should be pharmaceutically acceptable. Pharmaceutically acceptable salts include those described by Berge, Bighley and Monkhouse, J. Pharm. Sci., 1977, 66, 1-19. The term “pharmaceutically acceptable salts” includes salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, trishydroxylmethyl amino methane, tripropyl amine, tromethamine, and the like. When a compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Examples of pharmaceutically acceptable salts include the ammonium, calcium, magnesium, potassium, and sodium salts, and those formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The compounds of formula (I) may be prepared in crystalline or non-crystalline form, and, if crystalline, may optionally be solvated, e.g. as the hydrate. This invention includes within its scope stoichiometric solvates (e.g. hydrates) as well as compounds containing variable amounts of solvent (e.g. water).

Certain compounds of formula (I) are capable of existing in stereoisomeric forms (e.g. diastereoisomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The subject invention also includes isotopically-labeled compounds, which are identical to those recited in formula (I) and following, but for the fact that one or more atoms are replaced by an atom having an atomic mass or mass number different from the atomic mass or mass number usually predominating. Examples of isotopes that can be incorporated into compounds of the invention include isotopes of hydrogen, carbon, nitrogen, oxygen, phosphorous, fluorine, iodine, and chlorine, such as 3H, 11C, 14C, 18F, 123I and 125I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as 3H, 14C are incorporated, are useful in drug and/or substrate tissue distribution assays. Tritiated, i.e., 3H, and carbon-14, i.e., 14C, isotopes are particularly preferred for their ease of preparation and detectability. 11C and 18F isotopes are particularly useful in PET (positron emission tomography), and 125I isotopes are particularly useful in SPECT (single photon emission computerized tomography), all useful in brain imaging. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically labeled compounds of formula (I) and following of this invention can generally be prepared by carrying out the procedures disclosed in the Schemes and/or in the Examples below, by substituting a readily available isotopically labeled reagent for a non-isotopically labeled reagent.

The present invention also provides a process for the preparation of a compound of formula (I) or a pharmaceutically acceptable salt thereof, which process comprises:

(a) reacting a compound of formula (II),

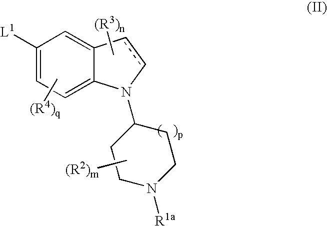

wherein R^{1a} is as defined for R¹ or an N-protecting group, R², R³, R⁴, m, n, p, q and [symbol] are as defined above and L¹ represents a suitable leaving group such as a halogen atom (e.g. bromo or iodo) or trifluoromethylsulfonyloxy, with a compound of formula A-SO₂—H (or A-SH followed by a subsequent oxidation step), wherein A is as defined above and thereafter as necessary removing an R^{1a} N-protecting group;
(b) deprotecting a protected derivative of a compound of formula (I); and thereafter optionally:
(c) interconversion to other compounds of formula (I) and/or forming a pharmaceutically acceptable salt and/or solvate.
(d) metallation of a compound of formula (II) followed by reaction with an appropriate arylsulfonylating electrophile to form a compound of formula (I), followed by process (b) or (c) as necessary.

Process (a) wherein a compound of formula (II) is treated with a compound of formula A-SO₂H typically comprises use of basic conditions and may be most conveniently carried out by using a suitable salt of the compound A-SO₂H (e.g. the sodium salt) in an appropriate solvent such as dimethyl sulfoxide, in the presence of a transition metal salt such as copper (I) iodide and a suitable additive such as N,N′-dimethylethylenediamine.

Process (b) wherein a compound of formula (II) is treated with a compound of formula A-SH typically comprises use of basic conditions e.g. by using a suitable salt of the compound A-SH (e.g. the sodium salt) in an appropriate solvent such as N,N-dimethylformamide, in the presence of a suitable metal salt such as copper (I) iodide, followed by use of a suitable oxidant such as 3-chloroperbenzoic acid, peracetic acid, magnesium monoperoxyphthalate or potassium monopersulfate. Alternatively, the compound of formula (II) can be advantageously treated with a compound of formula A-SH in the presence of a base such as potassium tert-butoxide in an appropriate solvent such as toluene in the presence of a suitable metal catalyst, e.g. a mixture of an appropriate palladium source such as tris(dibenzylideneacetone)dipalladium(0) and an appropriate ligand such as (oxydi-2,1-phenylene)-bis(diphenylphosphine), followed by oxidation as described above.

In processes (a) and (b), examples of protecting groups and the means for their removal can be found in T. W. Greene ‘Protective Groups in Organic Synthesis’ (J. Wiley and Sons, 1991). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid. A further amine protecting group includes methyl which may be removed using standard methods for N-dealkylation (e.g. 1-chloroethyl chloroformate under basic conditions followed by treatment with methanol).

Process (c) may be performed using conventional interconversion procedures such as epimerisation, oxidation, reduction, reductive alkylation, alkylation, nucleophilic or electrophilic aromatic substitution, ester hydrolysis or amide bond formation. For example, N-dealkylation of a compound of formula (I) wherein R¹ represents an alkyl group to give a compound of formula (I) wherein R¹ represents hydrogen. It will be appreciated that such interconversion may be interconversion of protected derivatives of formula (I) which may subsequently be deprotected following interconversion.

In addition, process (c) may comprise, for example, reacting a compound of formula (I), wherein R¹ represents hydrogen, with an aldehyde or ketone in the presence of a reducing agent in order to generate a compound of formula (I) where R¹ represents C₁₋₆ alkyl. This may be performed using a hydride donor agent such as sodium cyanoborohydride, sodium triacetoxyborohydride or a resin bound form of cyanoborohydride in an alcoholic solvent such as ethanol and in the presence of an acid such as acetic acid, or under conditions of catalytic hydrogenation. Alternatively, such a transformation may be carried out by reacting a compound of formula (I), wherein R¹ represents hydrogen, with a compound of formula R¹-L, wherein R¹ is as defined above and L represents a leaving group such as a halogen atom (e.g. bromine or iodine) or methylsulfonyloxy group, optionally in the presence of a suitable base such as potassium carbonate or triethylamine using an appropriate solvent such as N,N-dimethylformamide or a C₁₋₄alkanol.

Process (d) may comprise, for example, reacting a compound of formula (II) with a metallating agent such as sec- or tert-butyl lithium in a suitable solvent such as tetrahydrofuran to form an anion which can be reacted with an appropriate electrophile such as an arylsulfonyl fluoride to form a compound of formula (I). Arylsulfonyl fluorides may be conveniently prepared by the reaction of the corresponding arylsulfonyl chloride with a source of fluoride such as calcium and/or potassium fluoride in a suitable solvent such as acetonitrile, optionally in the presence of water or a crown ether.

Compounds of formula (II) wherein [symbol] represents a single bond may be prepared in accordance with the following scheme:

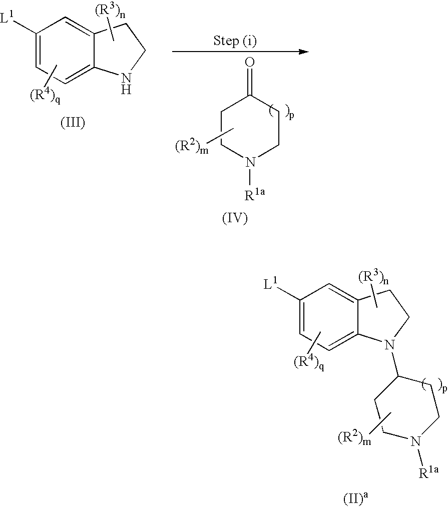

wherein R^{1a}, R², R³, R⁴, m, n, p, q and L¹ are as defined above.

Step (i) may typically be effected using a reducing agent such as sodium cyanoborohydride or sodium triacetoxyborohydride in a suitable solvent such as ethanol or 1,2-dichloroethane.

Compounds of formula (II) in which [symbol] represents a double bond may be prepared from compounds of formula (II)^{a} above by reaction with a suitable oxidising agent such as dichlorodicyano-1,4-benzoquinone in a suitable solvent such as tetrahydrofuran.

Compounds of formula (III) and (IV) are known in the literature or can be prepared by analogous methods.

Alternatively compounds of formula (I) wherein [symbol] represents a single bond may be prepared in accordance with the following scheme:

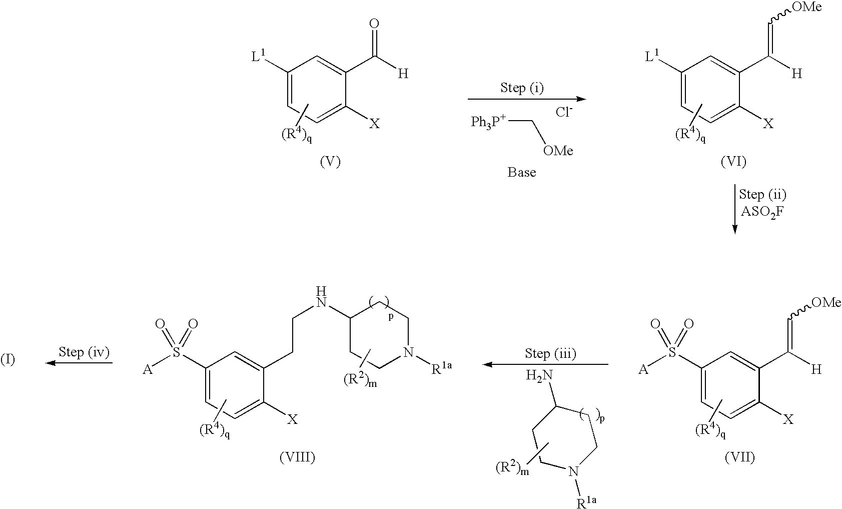

wherein A, R^{1a}, R², R³, R⁴, m, n, p, q and L¹ are as defined above and X is a suitable leaving group such as a halogen, for example fluorine, or an O-trifluoromethanesulfonate.

Step (i) may typically be effected using a Wittig agent such as [(methyloxy)methyl](triphenyl)phosphonium chloride in the presence of a suitable base such as 2-tert-butlyimino-2-diethylamino-1,3-dimethyl-perhydro-1,3,2-diazaphosphorine, which may conveniently be in a polymer bound form, in a solvent such as acetonitrile. Step (ii) can be effected by the metallation of (VI) using for example tert-butyllithium in a solvent such as tertrahydrofuran followed by reaction with a sulfonyl electrophile such as phenylsulfonyl fluoride. Step (iii) comprises the hydrolysis of the vinyl ether of (VII) using a suitable acid, such as formic acid, followed by reductive amination of the intermediate aldehyde with an appropriate amine such as 4-amino-1-Boc-piperidine in the presence of a suitable reducing agent, for example sodium triacetoxyborohydride, in an appropriate solvent such as 1,2-dichloroethane and in the presence of an acid catalyst such as acetic acid. Step (iv) can typically be effected by heating compound (VIII), optionally in the presence of a suitable organic or inorganic base, in a suitable solvent such as DMSO, or may be achieved using palladium catalysis in the presence of a suitable ligand.

In the case of compounds of formula (I) wherein [symbol] represents a single bond, n represents 2, and R³ and R^{3′} represent a 3,3-dialkyl substitution of the indoline ring, an additional alternative process is indicated in the following scheme:

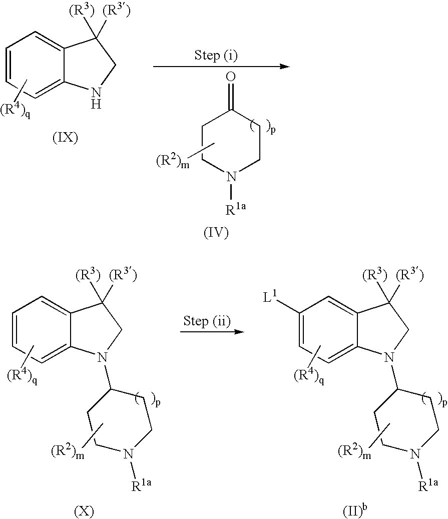

wherein R^{1a}, R², R³, R⁴, m, n, p, q and L₁ are as defined above and R³, is defined as for R³ but need not be identical to R³, and compounds of formula (II)^{b} are embodiments of formula (II).

Step (i) typically comprises the reaction of a compound of formula (IX), such as 3,3-dimethylindoline with an appropriate ketone such as N-Boc-piperidin-4-one in the presence of an appropriate reducing agent such as sodium cyanoborohydride in an appropriate solvent such as acetic acid. Step (ii) comprises the introduction of a leaving group L₁, for example iodine, using an electrophilic agent such as benzyltrimethylammonium dichloroiodate in a suitable solvent mixture such as dichloromethane and methanol and in the presence of an appropriate base such as calcium carbonate.

An alternative procedure for the preparation of compounds of formula (II) in which R⁴ is a halogen (Y) is shown in the following scheme:

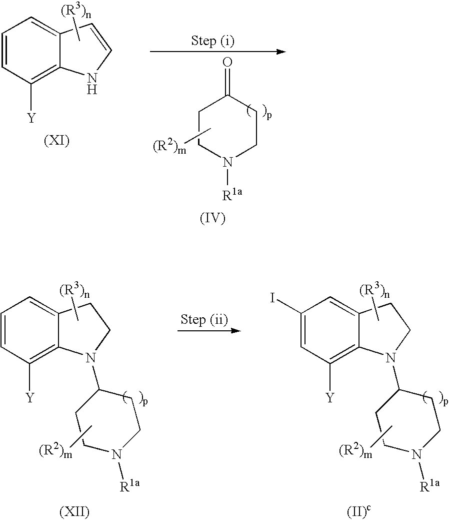

Step (i) typically comprises the reaction of a compound of formula (XI) such as 7-fluoroindole with a suitable ketone such as N-Boc-piperidin-4-one in the presence of a reducing agent, for example sodium cyanoborohydride and in a suitable solvent such as acetic acid to form (XII). Step (ii) comprises the reaction of (XII) with an electrophilic halogenating agent such as N-iodosuccinimide in an appropriate solvent such as Dimethylformamide to give a compound of formula (II)^{c}

Indolyl compounds of formula (I) in which R³=3-alkyl may be prepared by the reaction of the corresponding compounds in which R³═H and R^{1a} is a protecting group such as Boc with a suitable electrophile such as Eschenmoser's salt, followed by hydrogenation and deprotection and further elaboration of group R^{1a} as specified earlier.

Pharmaceutically acceptable salts may be prepared conventionally by reaction with the appropriate acid or acid derivative.

Compounds of formula (I) and their pharmaceutically acceptable salts have affinity for the 5-HT₆ receptor and are believed to be of potential use in the treatment of certain CNS disorders such as anxiety, depression, epilepsy, obsessive compulsive disorders, migraine, cognitive memory disorders (e.g. Alzheimer's disease, age related cognitive decline, mild cognitive impairment and vascular dementia), Parkinson's Disease, ADHD (Attention Deficit Disorder/Hyperactivity Syndrome), sleep disorders (including disturbances of Circadian rhythm), feeding disorders such as anorexia and bulimia, panic attacks, withdrawal from drug abuse such as cocaine, ethanol, nicotine and benzodiazepines, schizophrenia (in particular cognitive deficits of schizophrenia), stroke and also disorders associated with spinal trauma and/or head injury such as hydrocephalus. Compounds of the invention are also expected to be of use in the treatment of certain GI (gastrointestinal) disorders such as IBS (Irritable Bowel Syndrome). Compounds of the invention are also expected to be of use in the treatment of obesity.

Thus the invention also provides a compound of formula (I) or a pharmaceutically acceptable salt thereof, for use as a therapeutic substance, in particular in the treatment or prophylaxis of the above disorders. In particular the invention provides for a compound of formula (I) or a pharmaceutically acceptable salt thereof, for use in the treatment of depression, anxiety, Alzheimer's disease, age related cognitive decline, ADHD, obesity, mild cognitive impairment, schizophrenia, cognitive deficits in schizophrenia and stroke.

The invention further provides a method of treatment or prophylaxis of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for use in the treatment or prophylaxis of the above disorders.

5-HT₆ antagonists have the potential to be capable of increasing basal and learning-induced polysialylated neuron cell frequency in brain regions such as the rat medial temporal lobe and associated hippocampus, as described in WO 03/066056. Thus, according to a further aspect of the present invention, we provide a method of promoting neuronal growth within the central nervous system of a mammal which comprises the step of administering a compound of formula (I) or a pharmaceutically acceptable salt thereof.

In order to use the compounds of formula (I) in therapy, they will normally be formulated into a pharmaceutical composition in accordance with standard pharmaceutical practice.

The present invention also provides a pharmaceutical composition, which comprises a compound of formula (I) or a pharmaceutically acceptable salt thereof, and a pharmaceutically acceptable carrier.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, more particularly from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 200 mg, for example 20 to 40 mg; and such unit doses will preferably be administered once a day, although administration more than once a day may be required; and such therapy may extend for a number of weeks or months.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

### DESCRIPTION 1

### 1,1-Dimethylethyl 4-(5-bromo-2,3-dihydro-1H-indol-1-yl)-1-piperidinecarboxylate (D1)

### Case (i)

A solution of 5-bromoindoline (2.65 g, 13.4 mmol) in glacial acetic acid (30 ml) was treated with 1,1-dimethylethyl 4-oxo-1-piperidinecarboxylate (2.93 g, 14.7 mmol), maintaining the temperature below 30° C. This solution was treated by portionwise addition of sodium triacetoxyborohydride (4.25 g, 20 mmol) maintaining the temperature below 35° C. The mixture was then stirred at ambient temperature for one hour, poured into a mixture of ethyl acetate (200 ml) and water (100 ml), and neutralised to pH ˜8 by dropwise addition of 6M NaOH solution, while maintaining the temperature below 35° C. The organic phase was separated and the aqueous phase washed with ethyl acetate (50 ml). The combined organic phases were dried over magnesium sulphate, concentrated in vacuo and purified by chromatography on silica gel, eluting with an increasing proportion of ethyl acetate in hexane. This afforded 1,1-dimethylethyl 4-(5-bromo-2,3-dihydro-1H-indol-1-yl)-1-piperidinecarboxylate (D1) as a pale yellow oil (4.5 g, 90%): ¹H NMR (CDCl₃): δ 1.47 (9H, s), 1.53 (2H, ddd), 1.76 (2H, br.d), 2.76 (2H, br.t), 2.93 (2H, t), 3.35 (2H, t), 3.44 (1H, tt), 4.24 (2H, br.d), 6.26 (1H, s), 7.11 (1H, d), 7.12 (1H, s)

### Case (ii)

5-Bromoindoline (16.51 g, 83.4 mmol) was dissolved in AcOH (300 ml) in a 3-necked RB flask under argon. 1,1-Dimethylethyl 4-oxo-1-piperidinecarboxylate (20.34 g, 100 mmol) was added at once and the mixture was allowed to stir for 3-5 minutes after which NaBH(OAc)₃ (27.9 g, 125 mmol) was added portionwise. After 1 hour at room temperature the volume was reduced under reduced pressure to almost completion and the residue was redissolved in EtOAc (300 ml), washed with sat. NaHCO₃ (3×300 ml), brine (300 ml) and dried over MgSO₄. This solution was filtered and concentrated to afford 35 g of crude material that was subsequently purified by flash chromatography (Biotage 75+M cartridge) with a gradient of EtOAc (0-30%) in hexane. The 1,1-dimethylethyl 4-(5-bromo-2,3-dihydro-1H-indol-1-yl)-1-piperidinecarboxylate (D1) was isolated with 96% yield (30.5 g), consistent spectroscopically with that prepared in Case (i).

### DESCRIPTION 2

### 1,1-Dimethylethyl 4-(5-bromo-1H-indol-1-yl)-1-piperidinecarboxylate (D2)

1,1-Dimethylethyl 4-(5-bromo-2,3-dihydro-1H-indol-1-yl)-1-piperidinecarboxylate (D1) (2.0 g, 5.2 mmol) was dissolved in THF (20 ml) and cooled to 0° C. To this solution was added dropwise a solution of 2,3-dichloro-5,6-dicyano-1,4-benzoquinone (1.13 g, 5.7 mmol) in THF (10 ml) maintaining the temperature <10° C. The mixture turned black and was stirred at <10° C. for 1 hour. Ethyl acetate (80 ml) was added, the mixture washed with saturated aqueous sodium bicarbonate (4×50 ml), brine (50 ml), dried over magnesium sulfate and concentrated to a brown oil. This was purified using silica gel chromatography, eluting with an increasing proportion of ethyl acetate in pentane, to afford 1,1-dimethylethyl 4-(5-bromo-1H-indol-1-yl)-1-piperidinecarboxylate (D2) as a white solid (1.8 g, 91%):

¹H NMR (CDCl₃) [symbol] 1.49 (9H, s), 1.88 (2H, ddd) 2.06 (2H, d), 2.91 (2H, br.t), 4.28-4.35 (3H, m), 6.46 (1H, d), 7.17 (1H, d), 7.23-7.29 (2H, m), 7.75 (1H, d); MS: m/z (M+H)⁺ 379, 381; C₁₈H₂₃BrN₂O₂ requires: 378, 380

### DESCRIPTION 3

### Case (i)

### 1,1-Dimethylethyl 4-[5-(phenylsulfonyl)-1H-indol-1-yl]-1-piperidinecarboxylate (D3)

A mixture of 1,1-dimethylethyl 4-(5-bromo-1H-indol-1-yl)-1-piperidinecarboxylate (D2) (4.0 g, 11.7 mmol), sodium phenylsulfinate (5.0 g 35 mmol), copper (I) iodide (220 mg, 1.2 mmol), N,N′-dimethyl-ethylenediamine (0.25 ml, 2.3 mmol) and potassium carbonate (3.2 g, 23 mmol) was suspended in dimethyl sulfoxide (20 ml) and heated to 100° C. under an argon atmosphere for 18 hours. The mixture was cooled, dichloromethane (200 ml) added and water (100 ml) introduced. The organic phase was separated, dried over magnesium sulfate, evaporated, and the residue purified by flash chromatography on silica gel eluting with an increasing proportion of ethyl acetate in pentane to afford 1,1-dimethylethyl 4-[5-(phenylsulfonyl)-1H-indol-1-yl]-1-piperidinecarboxylate (D3) (1.6 g, 31%) as a white solid:

¹H NMR (CDCl₃) [symbol] 1.49 (9H, s), 1.87 (2H, ddd) 2.02 (2H, br.d), 2.91 (2H, br.t), 4.31-4.43 (3H, m), 6.66 (1H, d), 7.29 (1H, d), 4.41-7.48 (4H, m), 7.75 (1H, dd, 7.49-7.79 (2H, m), 8.3 (1H, d).

### Case (ii)

A solution of 2,3-dichloro-5,6-dicyano-p-benzoquinone (342 mg, 1.5 mmol) in THF (2 ml) was added dropwise to a stirred solution of 1,1-dimethylethyl 4-[5-(phenylsulfonyl)-2,3-dihydro-1H-indol-1-yl]-1-piperidinecarboxylate (D4) (434 mg, 0.983 mmol) in THF (13 ml) at 0° C. under argon. The reaction solution was stirred at 0° C. for 2 h followed by 3 h at RT. It was then concentrated and partitioned between EtOAc and dilute aqueous NaHCO₃ solution. The organic layer was separated and washed with dilute aqueous NaHCO₃ solution (×1) and brine (×1) before being dried over MgSO₄. It was filtered and evaporated to afford the crude product (457 mg). This was purified on silica (20 g) eluting with 40% EtOAc in hexane to give the pure title compound (414 mg, 94%); consistent spectroscopically with the material produced in Case (i).

### DESCRIPTION 4

### 1,1-Dimethylethyl 4-[5-(phenylsulfonyl)-2,3-dihydro-1H-indol-1-yl]-1-piperidinecarboxylate (D4)

### Case (i)

A mixture of 1,1-dimethylethyl 4-(5-bromo-2,3-dihydro-1H-indol-1-yl)-1-piperidinecarboxylate (D1) (0.7 g, 1.8 mmol), sodium phenylsulfinate (0.9 g, 5.5 mmol), copper (I) iodide (35 mg, 0.18 mmol), N,N′-dimethyl-ethylenediamine (0.04 ml, 0.36 mmol), and potassium carbonate (0.5 g, 3.6 mmol) was suspended in dimethyl sulfoxide (20 ml) and heated under an argon atmosphere to 100° C. for 18 hours. The mixture was cooled, dichloromethane (50 ml) added and water (25 ml) introduced. The organic phase was separated, dried over magnesium sulfate, evaporated, and the residue purified by flash chromatography on silica gel eluting with an increasing proportion of ethyl acetate in pentane to afford 1,1-dimethylethyl 4-[5-(phenylsulfonyl)-2,3-dihydro-1H-indol-1-yl]-1-piperidinecarboxylate (D4), as a white solid (238 mg, 28%):

¹H NMR (CDCl₃) [symbol] 1.46 (9H, s), 1.58 (2H, dt), 1.74 (2H, br.d), 2.77 (2H, br.t), 2.98 (2H, t), 3.50 (3H, t), 4.24 (2H, br.d), 6.40 (1H, d), 7.41-7.53 (4H, m), 7.66 (1H, dd), 7.90 (2H, dd); MS: m/z (M+H)⁺ 443; C₂₄H₃₀SN₂O₄ requires: 442.

### Case (ii)

In an oven dried 500 ml 3-necked RB flask equipped with a temperature probe and a 50 ml dropping funnel was placed 1,1-dimethylethyl 4-(5-bromo-2,3-dihydro-1H-indol-1-yl)-1-piperidinecarboxylate (D1) (10 g, 26.3 mmol) under argon and it was then dissolved in dry THF (130 ml). This solution was cooled to −78° C. and tert-butyllithium (35 ml, 52.5 mmol, 2 eq, 1.5M in pentane) was added dropwise (keeping the temperature below −60° C.). After the addition was complete the bright yellow solution was stirred at −70° C. for 5 minutes and then a solution of benzenesulfonylfluoride (6.32 g, 39.45 mmol) in dry THF (30 ml) was added over a period of 15 minutes. The resulting brown solution was stirred at −70° C. for 15 minutes and then it was allowed to warm to room temperature. After 1 hour it was quenched with sat. NH₄Cl, extracted with EtOAc, the organics washed with brine and dried over MgSO₄. The crude material (14.5 g) was purified by flash chromatography (Biotage 75+S cartridge) with a gradient of EtOAc in Hexane to afford 1,1-dimethylethyl 4-[5-(phenylsulfonyl)-2,3-dihydro-1H-indol-1-yl]-1-piperidinecarboxylate (D4) in 56% yield (6.5 g), consistent spectroscopically with that prepared in Case (i).

### DESCRIPTION 5

### 3,3-Dimethyl-1-(1-methyl-4-piperidinyl)-2,3-dihydro-1H-indole (D5)

To a solution of 3,3-dimethyl-2,3-dihydro-1H-indole (500 mg, 3.4 mmol) in acetic acid (5 ml) under an argon atmosphere was added 1-methyl-4-piperidinone (423 mg, 3.74 mmol) and after 5 minutes NaBH(OAc)₃ (1.08 g, 5.1 mmol) was added in one portion. After 0.5 hours the mixture was diluted with water, basified with NaOH (pellets) until pH ca. 10 and extracted with Et₂O. The organic phase was separated and dried over MgSO₄. The solution was filtered and concentrated to afford 3,3-dimethyl-1-(1-methyl-4-piperidinyl)-2,3-dihydro-1H-indole (D5) in 94% yield (780 mg).

¹H-NMR (CDCl₃): δ1.27 (6H, s), 1.74 (4H, m), 2.04 (2H, dt), 2.30 (3H, s), 2.95 (2H, d), 3.13 (2H, s), 3.35 (1H, m), 6.40 (1H, d, J=7.6 Hz), 6.63 (1H, t, J=7.6 Hz), 6.98 (1H, d, J=7.6 Hz), 7.04 (1H, t, J=7.6 Hz).

MS: m/z (M+H)⁺ 245, C₁₆H₂₄N₂ requires 244.

### DESCRIPTION 6

### 1,1-Dimethylethyl 4-(5-iodo-2,3-dihydro-1H-indol-1-yl)-1-piperidinecarboxylate (D6)

5-Iodoindole (12.15 g, 0.05 mol) in glacial acetic acid (150 ml) at RT was treated portionwise with sodium cyanoborohydride (9.42 g, 0.15 mol) over 10 minutes. The resulting solution was allowed to stir at RT for 1 hr. and it was then evaporated to near dryness (caution: evaporation to complete dryness causes rapid exothermic decomposition). The residue was diluted with EtOAc and washed with aqueous potassium carbonate solution and brine. Glacial acetic acid (50 ml) was added to the solution which was then dried (MgSO₄), filtered and evaporated to near dryness to leave the crude 5-iodoindoline solution. Further glacial acetic acid (120 ml) was then added followed by 1,1-dimethylethyl 4-oxo-1-piperidinecarboxylate (11.94 g, 0.06 mol). Sodium triacetoxyborohydride (15.94 g, 0.075 mol) was then added portionwise over 10 minutes and the reaction solution was stirred at RT for 1 hr. Further 1,1-dimethylethyl 4-oxo-1-piperidinecarboxylate (2.99 g, 0.015 mol) and sodium triacetoxyborohydride (5.3 g, 0.084 mol) were added and stirring continued for a further hour. The solution was then evaporated to near dryness and partitioned between EtOAc and aqueous potassium carbonate solution. The organic layer was separated and washed with aqueous potassium carbonate solution and brine. It was dried (MgSO₄), filtered and evaporated to leave the crude product. This was purified on silica eluting with hexane/EtOAc (9:1) to afford 1,1-dimethylethyl 4-(5-iodo-2,3-dihydro-1H-indol-1-yl)-1-piperidinecarboxylate (D6) as a white solid (15.20 g, 71%).

¹H NMR (CDCl₃) δ 1.47 (9H, s), 1.55 (2H, m), 1.75 (2H, m), 2.76 (2H, m), 2.93 (2H, t J=8.6 Hz), 3.35 (2H, t J=8.6 Hz), 3.44 (1H, m), 6.19 (1H, d J=8.8 Hz) and 7.30 (2H, m).

MS (electrospray): m/z (M+H)⁺ 429; C₁₈H₂₅IN₂O₂ requires M=428.

### DESCRIPTION 7

### 5-Bromo-1-(octahydro-7-indolizinyl)-2,3-dihydro-1H-indole (D7)

To a solution of 5-bromoindoline (300 mg, 1.52 mmol) in acetic acid (3 ml) was added hexahydro-7(1H)-indolizinone (232 mg, 1.67 mmol) (prepared in a similar manner to J. Chem. Soc. Perkin Trans., 1986, 447-453) and after 5 minutes NaBH(OAc)₃ (480 mg, 2.28 mmol) was added portionwise. The mixture was left stirring at room temperature under argon and after 4 hours it was concentrated under reduced pressure, dissolved in dichloromethane and treated with a 5% K₂CO₃. the organic layer was separated, washed with brine and dried over MgSO₄. The crude material (447 mg) was purified by flash chromatography (Biotage, 20 g column) with a gradient of MeOH in dichloromethane to give 5-bromo-1-(octahydro-7-indolizinyl)-2,3-dihydro-1H-indole (D7) in 56% yield.

¹H-NMR (CDCl₃): δ 1.48 (1H, m), 1.59 (2H, m), 1.70 (1H, m), 1.85 (3H, m), 2.19 (1H, m), 2.30 (1H, m), 2.40 (2H, m), 2.90 (3H, m), 3.02 (1H, m), 3.5 (3H, m), 6.27 (1H, d, J=8.0 Hz), 7.11 (2H, m).

### DESCRIPTION 8

### 5-Iodo-3,3-dimethyl-1-(1-methyl-4-piperidinyl)-2,3-dihydro-1H-indole (D8)

3,3-Dimethyl-1-(1-methyl-4-piperidinyl)-2,3-dihydro-1H-indole (D5) (775 mg, 3.18 mmol) was dissolved in a mixture of CH₂Cl₂/MeOH (42 ml/16 ml) and CaCO₃ (414 mg, 4.13 mmol) and benzyltrimethylammonium dichloroiodate (95%) (1.16 g, 3.18 mmol) were added and the mixture was stirred at RT under argon. The reaction was followed by TLC/LC-MS. After 1 hour the excess CaCO₃ was filtered through celite, the solvent was evaporated under reduced pressure, the product redissolved in dichloromethane and washed with sodium thiosulphate (10 g, 10% w/v). The organic phase was dried over MgSO₄ and concentrated to afford a yellow solid 5-iodo-3,3-dimethyl-1-(1-methyl-4-piperidinyl)-2,3-dihydro-1H-indole (D8) in quantitative yield (1.2 g).

¹H-NMR (CDCl₃): δ1.25 (6H, s), 1.94 (2H, m), 2.38 (2H, m), 2.73 (3H, s), 2.75 (2H, m), 3.17 (2H, s), 3.52 (3H, m), 6.15 (1H, d, J=8.25 Hz), 7.22 (1H, s), 7.31 (1H, d, J=8.25 Hz).

MS: m/z (M+H)⁺ 371, C₁₆H₂₃IN₂ requires 370.

### DESCRIPTION 9

### 1,1-Dimethylethyl 4-(7-fluoro-2,3-dihydro-1H-indol-1-yl)-1-piperidinecarboxylate (D9)

Prepared from 7-fluoro-1H-indole and 1,1-dimethylethyl 4-oxo-1-piperidinecarboxylate in a similar manner to Description 6 with the addition that an extra 1.0 equivalents of sodium cyanoborohydride was added during the first reduction. 1,1-Dimethylethyl 4-(7-fluoro-2,3-dihydro-1H-indol-1-yl)-1-piperidinecarboxylate (D9) was obtained as a colourless oil (39%).

¹H-NMR (CDCl₃): δ 1.44 (9H, s), 1.57 (2H, m), 1.77 (2H, m), 2.76 (2H, m), 2.97 (2H, t, J=8.6 Hz), 3.35 (2H, t, J=8.6 Hz), 3.95 (1H, m), 4.22 (2H, m), 6.60 (1H, m), 6.75-6.91 (2H, m). MS: m/z (M-^{t}Bu+H)⁺ 265, C₁₈H₂₅FN₂O₂ requires 320.

### DESCRIPTION 10

### 1,1-Dimethylethyl 3-(5-iodo-2,3-dihydro-1H-indol-1-yl)-1-pyrrolidinecarboxylate (D10)

Prepared from 5-Iodo-1H-Indole and 1,1-Dimethylethyl 3-Oxo-1-Pyrrolidinecarboxylate in a similar manner to Description 6. 1,1-Dimethylethyl 3-(5-iodo-2,3-dihydro-1H-indol-1-yl)-1-pyrrolidinecarboxylate was obtained as a yellow oil (D10) (51%).

¹H-NMR (CDCl₃): δ 1.46 (9H, s), 1.98-2.15 (2H, m), 2.94 (2H, t, J=8.2 Hz), 3.28-3.67 (6H, m), 4.08 (1H, m), 6.26 (1H, d, J=4.2 Hz), 7.32 (2H, m). MS: m/z (M-^{t}Bu+H)⁺ 359, C₁₇H₂₃IN₂O₂ requires 414.

### DESCRIPTION 11

### 1,1-Dimethylethyl 4-(7-fluoro-5-iodo-2,3-dihydro-1H-indol-1-yl)-1-piperidinecarboxylate (D11)

A solution of 1,1-dimethylethyl 4-(7-fluoro-2,3-dihydro-1H-indol-1-yl)-1-piperidinecarboxylate (D9) (1.09 g, 3.40 mmol, 1.0 equivalents) and N-iodosuccinimide (0.919 g, 4.08 mmol, 1.2 equivalents) in dry dimethylformamide (33 ml) was stirred under argon in the dark for 4 h. At this point additional N-iodosuccinimide (230 mg, 1.02 mmol, 0.3 equivalents) was added and the solution stirred for a further 1 h. The dimethylformamide was then evaporated and the resulting green oil partitioned between ethyl acetate (100 ml) and saturated aqueous sodium bicarbonate (75 ml). The organic phase was separated, washed with saturated aqueous sodium bicarbonate (50 ml) and then brine (50 ml), dried with magnesium sulphate, filtered, and evaporated to dryness. The resulting brown oil (1.72 g) was purified on silica eluting with pentane and ethyl acetate (0 to 90%). The appropriate fractions were combined and evaporated to dryness, producing a yellow oil (594 mg), which was then purified by mass directed preparative HPLC. 1,1-Dimethylethyl 4-(7-fluoro-5-iodo-2,3-dihydro-1H-indol-1-yl)-1-piperidinecarboxylate (D11) was obtained as a colourless oil (228 mg, 19%).

¹H-NMR (CDCl₃): δ 1.46 (9H, s), 1.53 (2H, m), 1.74 (2H, m), 2.76 (2H, m), 2.96 (2H, t, J=8.6 Hz), 3.36 (2H, t, J=8.6 Hz), 3.88 (1H, m), 4.15 (2H, m), 7.07-7.11 (2H, m). MS: m/z (M-^{t}Bu+H)⁺ 391, C₁₈H₂₄FIN₂O₂ requires 446.

### DESCRIPTION 12

### 1,1-Dimethylethyl 4-(7-chloro-5-iodo-2,3-dihydro-1H-indol-1-yl)-1-piperidinecarboxylate (D12)

A solution of 1,1-dimethylethyl 4-(5-iodo-2,3-dihydro-1H-indol-1-yl)-1-piperidinecarboxylate (D6) (1.00 g, 2.34 mmol, 1.0 equivalents), and N-chlorosuccinimide (0.374 g, 2.80 mmol, 1.2 equivalents) in dry dimethylformamide (30 ml) was stirred under argon in the dark for 4 h. The dimethylformamide was then evaporated and the resulting green oil partitioned between ethyl acetate (50 ml) and saturated aqueous sodium bicarbonate (50 ml). The organic phase was separated, washed with saturated aqueous sodium bicarbonate (20 ml) and then brine (20 ml), dried with magnesium sulphate, filtered, and evaporated to dryness. The resulting green foam (1.18 g) was purified using mass directed preparative HPLC. 1,1-Dimethylethyl 4-(7-chloro-5-iodo-2,3-dihydro-1H-indol-1-yl)-1-piperidinecarboxylate (D12) was obtained as a green oil (525 mg, 49%).

¹H-NMR (CDCl₃): δ 1.46 (9H, s), 1.55 (2H, m), 1.73 (2H, m), 2.76 (2H, m), 2.92 (2H, t, J=8.6 Hz), 3.42 (2H, t, J=8.6 Hz), 4.21 (2H, m), 4.45 (1H, m), 7.18 (1H, m), 7.28, 1H, m).

MS: m/z (M+H)⁺ 463 & 465, C₁₈H₂₄ClIN₂O₂ requires 462 & 464.

### DESCRIPTION 13

### 1,1-Dimethylethyl 4-(5-iodo-2,3-dihydro-1H-indol-1-yl)hexahydro-1H-azepine-1-carboxylate (D13)

Prepared from 5-iodo-1H-indole and 1,1-dimethylethyl 4-oxohexahydro-1H-azepine-1-carboxylate in a similar manner to description Description 6,1,1-Dimethylethyl 4-(5-iodo-2,3-dihydro-1H-indol-1-yl)hexahydro-1H-azepine-1-carboxylate (D13) was obtained as a yellow oil (62%).

¹H-NMR (CDCl₃): δ 1.50 (9H, s), 1.50-1.99 (6H, m), 2.90 (2H, m), 3.22-4.15 (7H, m), 6.15 (1H, d, J=8.8 Hz), 7.26-7.31 (2H, m). MS: m/z (M-^{t}Bu+H)⁺ 387, C₁₉H₂₇IN₂O₂ requires 442.

### DESCRIPTION 14

### 4-Fluorobenzenesulfonyl fluoride (D14)

4-Fluorobenzenesulfonyl chloride (1.46 g, 7.5 mmol, 1.0 equivalents), potassium fluoride (2.18 g, 37.5 mmol, 5 equivalents) and 18 crown 6 (50 mg) were stirred in acetonitrile (15 ml), at room temperature, under argon, for 5 h. Saturated aqueous sodium bicarbonate (50 ml) was added and then the mixture was extracted with ethyl acetate (2×50 ml). The organic extracts were combined, washed with saturated aqueous sodium bicarbonate (25 ml), dried with magnesium sulphate, filtered and evaporated to dryness. 4-Fluorobenzenesulfonyl fluoride (D14) was obtained as a yellow oil (1.25, 94%).

¹³C-NMR (CDCl₃): δ 117.3 (d, J=30 Hz), 129.0 (m), 131.6 (d, J=10 Hz), 166.9 (d, J=260 Hz).

### DESCRIPTION 15

### 3-Fluorobenzenesulfonyl fluoride (D15)

3-Fluorobenzenesulfonyl chloride (2.92 g, 15 mmol, 1 equivalent), potassium fluoride (4.36 g, 75 mmol, 5 equivalents) and water (1 drop) were stirred in acetonitrile (60 ml), at room temperature, under argon, for 16 h. The mixture was filtered, evaporated to dryness and the oily residues partitioned between ethyl acetate (20 ml) and saturated aqueous sodium bicarbonate (20 ml). The organic phase was separated washed with saturated aqueous sodium bicarbonate (20 ml) and then brine (20 ml), dried with magnesium sulphate, filtered and evaporated to dryness. 3-Fluorobenzenesulfonyl fluoride (D15) was obtained as a yellow oil (2.24 g, 84%).

¹³C-NMR (CDCl₃): δ 115.9 (d, J=20 Hz), 123.1 (d, J=30 Hz), 124.4 (d, J=10 Hz), 131.7 (d, J=10 Hz), 134.7 (m), 162.4 (d, J=250 Hz).

### DESCRIPTION 16

### 2-Cyanobenzenesulfonyl fluoride (D16)

See Table 1.

### DESCRIPTION 17

### 3-Cyanobenzenesulfonyl fluoride (D17)

See Table 1.

### DESCRIPTION 18

### 3,4-Difluorobenzenesulfonyl fluoride (D18)

See Table 1.

### DESCRIPTION 19

### 2,5-Difluorobenzenesulfonyl fluoride (D19)

See Table 1.

### DESCRIPTION 20

### 2-Methylbenzenesulfonyl fluoride (D20)

See Table 1.

### DESCRIPTION 21

### 3-Methylbenzenesulfonyl fluoride (D21)

See Table 1.

### DESCRIPTION 22

### 4-Methylbenzenesulfonyl fluoride (D22)

See Table 1.

### DESCRIPTION 23

### 1,3,5-Trimethyl-1H-pyrazole-4-sulfonyl fluoride (D23)

See Table 1.

### DESCRIPTION 24

### 2,4-Dimethyl-1,3-thiazole-5-sulfonyl fluoride (D24)

See Table 1.

### DESCRIPTION 25

### 3-(Methyloxy)benzenesulfonyl fluoride (D25)

See Table 1.

Description 26

### i) 1,1-Dimethylethyl 4-methyl-4-({[(phenylmethyl)oxy]carbonyl}amino)-1-piperidinecarboxylate (D26)

To a solution of 1-{[(1,1-dimethylethyl)oxy]carbonyl}-4-methyl-4-piperidinecarboxylic acid (1.02 g, 4.2 mmol), benzyl alcohol (0.91 g, 8.4 mmol) and triethylamine (0.47 g, 4.6 mmol) in toluene (10 ml) at RT was added diphenyl phosphoryl azide (1.27 g, 4.6 mmol). The solution was stirred at RT for 0.25 h and then heated to 90° C. for 2 h. After cooling to RT the solution was diluted with EtOAc and washed with dil. HCl soln., dil. NaHCO₃ soln. and brine. It was dried, filtered and evaporated to leave a colourless oil (1.74 g), which was purified on silica eluting with 0-15% EtOAc in hexane to afford 1,1-dimethylethyl 4-methyl-4-({[(phenylmethyl)oxy]carbonyl}amino)-1-piperidinecarboxylate (D26), the title compound (596 mg).

¹H NMR (CDCl₃) δ 1.38 (3H, s), 1.45 (9H, s), 1.54 (2H, m partially obscured by water), 1.95 (2H, m), 3.15 (2H, m), 3.62 (2H, br. m), 4.59 (1H, br.s), 5.06 (2H, s), and 7.45 (5H, m).

### DESCRIPTION 27

### 4-Bromo-1-fluoro-2-[2-(methoxy)ethenyl]benzene (D27)

A suspension of 5-bromo-2-fluorobenzaldehyde (3.2 g, 15 mmol) and [(methoxy)methyl](triphenyl)phosphonium chloride (8.5 g, 25 mmol), in 1,2-dichloroethane (50 ml), was treated with 2-tert-butylimino-2-diethylamino-1,3-dimethyl-perhydro-1,3,2-diazaphosphorine on polystyrene (12.5 g, approximately 25 mmol base equiv. by loading) and warmed to 75° C. for two hours. The mixture was cooled to 50° C. and stirred for a further 12 hours then warmed to 80° C. for 3 hours. The mixture was cooled, filtered and evaporated. The residue was dissolved in diethyl ether (50 ml), filtered and the ether evaporated to leave a residue which was subjected to purification by flash chromatography (Biotage FM2:70 g silica column, eluting with 100% pentane to 100% dichloromethane), to obtain 4-bromo-1-fluoro-2-[2-(methoxy)ethenyl]benzene (D27) as an approximately 1:1× mixture of E and Z isomers, as a colourless wax, 1.2 g. E-isomer ¹H NMR (CDCl₃) δ 3.71, (3H, s),), 5.76 (1H, d, J=13.2 Hz), 6.88 (1H, dd), 7.14 (1H, d, J=13.2 Hz), 7.16-7.19 (1H, m), 7.38 (1H, dd, J=2.4 Hz, J=6.8 Hz), Z-isomer ¹H NMR (CDCl₃) δ 3.82 (3H, s), 5.39 (1H, d, J=7.2 Hz, 6.26 (01H, d, J=7.2 Hz), 6.86 (1H, dd), 7.17-7.22 (1H, m), 8.17 (1H, dd, J=2.4, 6.8 Hz).

### DESCRIPTION 28

### 4-Fluoro-3-[2-(methoxy)ethenyl]phenyl phenyl sulfone (D28)

A solution of 4-bromo-1-fluoro-2-[(E)-2-(methoxy)ethenyl]benzene (D27), (1.15 g, 5 mmol), in THF (10 ml) was cooled to −78° C. under argon. A solution of tert-butyl lithium (1.5 M in pentane, 6.67 ml, 10 mmol) was added followed after five minutes by a solution of phenylsulfonyl fluoride (1.2 g, 7.5 mmol) in THF (5 ml). The mixture was stirred at −78° C. for 30 minutes then quenched by the addition of satd. aq. ammonium chloride solution (1 ml). The mixture was warmed to room temperature, evaporated and the residue subjected to purification by flash chromatography (eluting with 100% pentane to 100% ethyl acetate using Biotage flash silica), to obtain 4-fluoro-3-[2-(methoxy)ethenyl]phenyl phenyl sulfone (D28), as a clear oil approximately, 1.05 g (72%), approx 92% pure by LCMS.

(ES): m/z (M+H)⁺ 293; C₁₅H₁₃FO₃S requires M=292.

### DESCRIPTION 29

### 1,1-Dimethylethyl 4-amino-4-methyl-1-piperidinecarboxylate (D29)

1,1-Dimethylethyl 4-methyl-4-({[(phenylmethyl)oxy]carbonyl}amino)-1-piperidinecarboxylate (D26) (596 mg, 1.71 mmol) in ethanol (12 ml) was hydrogenated over 10% palladium on carbon (200 mg; 50% water) at ambient temperature and pressure for 42 h. The catalyst was filtered and the filtrate evaporated to dryness to afford 1,1-dimethylethyl 4-amino-4-methyl-1-piperidinecarboxylate (D29) as a white foam. This was used in the next step without purification.

¹H NMR (CDCl₃) δ 1.36 (3H, s), 1.45 (9H, s), 1.59 (2H, m), 1.72 (2, m), 3.37 (2H, m), 3.57 (2H, m) and 7.15-7.40 (5H, m).

### DESCRIPTION 30

### 1,1-Dimethylethyl 4-({2-[2-fluoro-5-(phenylsulfonyl)phenyl]ethyl}amino)-4-methyl-1-piperidinecarboxylate (D30)

A solution of crude 4-fluoro-3-[2-(methoxy)ethenyl]phenyl phenyl sulfone (D28) (300 mg, approx 1.05 mmol) was dissolved in formic acid (98%, 3 ml) and stood at RT for 16 hours, 1,2-dichloroethane (30 ml) was added and the mixture washed with saturated aqueous sodium acetate (10 ml). The DCE phase was separated and treated with acetic acid (3 drops, ˜50 mg), 1,1-dimethylethyl 4-amino-4-methyl-1-piperidinecarboxylate (D29) and sodium triacetoxyborohydride (2.1 mmol, 445 mg), and stirred at RT for 16 hours. The mixture was washed with satd. aq. sodium bicarbonate, dried (MgSO₄), filtered and evaporated and the residue purified by flash chromatography on Biotage aminated silica (eluting with hexane-ethyl acetate) followed by further purification on standard silica gel eluting with 0-2% methanol in dichloromethane to give 1,1-dimethylethyl 4-({2-[2-fluoro-5-(phenylsulfonyl)phenyl]-ethyl}amino)-4-methyl-1-piperidinecarboxylate (D30) 223 mg.

¹H NMR (CDCl₃) δ 1.03 (3H, s), 1.38 (4H, m), 1.45 (9H, s), 2.78 (4H, br. s), 3.20 (2H, m), 3.37 (2H, m), 7.13 (1H, t J=14.2 Hz), 7.51 (3H, m) 7.80 (1H, m) and 7.91 (3H, m). MS (electrospray): m/z (M+H)⁺ 477; C₂₅H₃₃FN₂O₄S requires M=476.

### DESCRIPTION 31

### 1,1-Dimethylethyl 4-methyl-4-[5-(phenylsulfonyl)-2,3-dihydro-1H-indol-1-yl]-1-piperidinecarboxylate (D31)

1,1-Dimethylethyl 4-({2-[2-fluoro-5-(phenylsulfonyl)phenyl]ethyl}amino)-4-methyl-1-piperidinecarboxylate (D30) (142 mg, 0.30 mmol) in DMSO (5 ml) was heated at 110° C. under argon for 3 days. The reaction solution was evaporated to dryness and the residue partitioned between EtOAc and dil. aq. K₂CO₃ soln. The organic phase was separated and washed with water and brine. It was dried over MgSO₄, filtered and evaporated to afford the crude product as a yellow oil (141 mg). This was purified on silica eluting with hexane/EtOAc 3:1 to 2:1 to afford a mixture of the title compound and corresponding indole in a ratio of ˜3:1. This (94 mg, 0.21 mmol) in DMF (5 ml) was treated with Eschenmoser's salt (20 mg, 0.11 mmol) and heated at 55° C. under argon. After 2 h more Eschenmoser's salt (60 mg, 0.33 mmol) was added and the temperature raised to 65° C. After a further 2 h the reaction solution was cooled and evaporated to dryness. The residue was partitioned between EtOAc and dil. aq. K₂CO₃ soln. The organic phase was separated and washed with water and brine. It was dried over MgSO₄, filtered and evaporated to afford the crude product as a colourless oil. This was purified on silica eluting with hexane/EtOAc 3:1 to afford the pure title compound 1,1-Dimethylethyl 4-methyl-4-[5-(phenylsulfonyl)-2,3-dihydro-1H-indol-1-yl]-1-piperidinecarboxylate (D31) (62 mg).

¹H NMR (CDCl₃) δ 1.26 (3H, s), 1.45 (9H, s), 1.66 (2H, m partially obscured by water), 2.12 (2H, m), 2.90 (2H, t J=8.2 Hz), 3.40 (2H, m), 3.50 (4H, m), 6.67 (1H, d J=8.4 Hz), 7.50 (4H, m), 7.60 (1H, m) and 7.90 (2H, m). MS (electrospray): m/z (M+H)⁺ 457; C₂₅H₃₂N₂O₄S requires M=456.

### DESCRIPTION 32

### 1,1-Dimethylethyl 3-[5-(phenylsulfonyl)-2,3-dihydro-1H-indol-1-yl]-1-pyrrolidinecarboxylate (D32)

Prepared from 1,1-dimethylethyl 3-(5-iodo-2,3-dihydro-1H-indol-1-yl)-1-pyrrolidinecarboxylate (D10) and sodium phenylsulfinate in a similar manner to Description 4 Case (i), heating at 110° C. for 16 h. 1,1-Dimethylethyl 3-[5-(phenylsulfonyl)-2,3-dihydro-1H-indol-1-yl]-1-pyrrolidinecarboxylate (D32) was obtained as a white solid (92%).

¹H-NMR (CDCl₃): δ 1.46 (9H, s), 2.04 (2H, m), 3.00 (2H, t, J=8.5 Hz), 3.40 (2H, m), 3.53 (4H, m), 4.15 (1H, m), 6.40 (1H, d, J=8.5 Hz), 7.48 (4H, m), 7.68 (1H, m), 7.90 (2H, m).

MS: m/z (M-^{t}Bu+H)⁺ 373, C₂₃H₂₈N₂O₄S requires 429.

### DESCRIPTION 33

### 1,1-Dimethylethyl 4-[5-(phenylsulfonyl)-2,3-dihydro-1H-indol-1-yl]hexahydro-1H-azepine-1-carboxylate (D33)

Prepared from 1,1-dimethylethyl 4-(5-iodo-2,3-dihydro-1H-indol-1-yl)hexahydro-1H-azepine-1-carboxylate (D13) and sodium phenylsulfinate in a similar manner to Description 4 (Case i), heating at 110° C. for 16 h. 1,1-Dimethylethyl 4-[5-(phenylsulfonyl)-2,3-dihydro-1H-indol-1-yl]hexahydro-1H-azepine-1-carboxylate was obtained as a white solid (D33) (71%).

¹H-NMR (CDCl₃): δ 1.48 (9H, s), 1.56-1.94 (6H, m), 2.96 (2H, m), 3.21-3.66 (7H, m), 6.28 (1H, d, J=8.4 Hz), 7.43-7.51 (4H, m), 7.65 (1H, m), 7.89 (2H, m), MS: m/z (M-^{t}Bu+H)⁺ 373, C₂₅H₃₂N₂O₄S requires 457.

### DESCRIPTION 34

### 1,1-Dimethylethyl 4-[7-fluoro-5-(phenylsulfonyl)-2,3-dihydro-1H-indol-1-yl]-1-piperidinecarboxylate (D34)

Prepared from 1,1-dimethylethyl 4-(7-fluoro-5-iodo-2,3-dihydro-1H-indol-1-yl)-1-piperidinecarboxylate (D11) and sodium phenylsulfinate in a similar manner to Description 4, heating at 110° C. for 16 h. 1,1-Dimethylethyl 4-[7-fluoro-5-(phenylsulfonyl)-2,3-dihydro-1H-indol-1-yl]-1-piperidinecarboxylate (D34) was obtained as a colourless oil (139 mg, 53%).

¹H-NMR (CDCl₃): δ 1.46 (9H, s), 1.58 (2H, m), 1.74 (2H, m), 2.74 (2H, m), 3.01 (2H, t, J=8.8 Hz), 3.51 (2H, t, J=9.0 Hz), 3.98 (1H, m), 4.13 (2H, m), 7.33 (1H, d, J=1.6 Hz), 7.40 (1H, dd, J=12 Hz & 1.8 hz), 7.46-7.55 (3H, m), 7.90 (2H, m).

MS: m/z (M-^{t}Bu+H)⁺ 405, C₂₄H₂₉FN₂O₄S requires 461.

### DESCRIPTION 35

### 1,1-Dimethylethyl 4-[7-chloro-5-(phenylsulfonyl)-2,3-dihydro-1H-indol-1-yl]-1-piperidinecarboxylate (D35)

Prepared from 1,1-dimethylethyl 4-(7-chloro-5-iodo-2,3-dihydro-1H-indol-1-yl)-1-piperidinecarboxylate (D12) and sodium phenylsulfinate in a similar manner to Description 4 (Case i), heating at 110° C. for 16 h. 1,1-Dimethylethyl 4-[7-chloro-5-(phenylsulfonyl)-2,3-dihydro-1H-indol-1-yl]-1-piperidinecarboxylate (D35) was obtained as a white solid (90%).

¹H-NMR (CDCl₃): δ 1.46 (9H, s), 1.61 (2H, m), 1.75 (2H, d, J=11.6), 2.75 (2H, m), 2.97 (2H, t, J=9.0 Hz), 3.53 (2H, t, J=9.0 Hz), 4.21 (2H, m), 4.65 (1H, m), 7.35 (1H, d, J=2.0 Hz), 7.47-7.61 (3H, m), 7.61 (1H, d, J=2.0 Hz), 7.90 (2H, m).

MS: m/z (M-^{t}Bu+H)⁺ 421 & 423, C₂₄H₂₉ClN₂O₄S requires 476 & 478.

### DESCRIPTION 36

### 1,1-Dimethylethyl 4-{5-[(4-fluorophenyl)sulfonyl]-2,3-dihydro-1H-indol-1-yl}-1-piperidinecarboxylate (D36)

Prepared from 1,1-dimethylethyl 4-(5-iodo-2,3-dihydro-1H-indol-1-yl)-1-piperidinecarboxylate (D6) and 4-fluorobenzenesulfonyl fluoride (D14) using n-butyl lithium (1.5 equivalents), in a similar manner to Description 45, 1,1-Dimethylethyl 4-{5-[(4-fluorophenyl)sulfonyl]-2,3-dihydro-1H-indol-1-yl}-1-piperidinecarboxylate (D36) was obtained as a yellow foam (48%).

MS: m/z (M-^{t}Bu+H)⁺ 405, C₂₄H₂₉FN₂O₄S requires 460.

### DESCRIPTION 37

### 1,1-Dimethylethyl 4-{5-[(2-cyanophenyl)sulfonyl]-2,3-dihydro-1H-indol-1-yl}-1-piperidinecarboxylate (D37)

Prepared from 1,1-dimethylethyl 4-(5-iodo-2,3-dihydro-1H-indol-1-yl)-1-piperidinecarboxylate (D6) and 2-cyanobenzenesulfonyl fluoride (D16) using t-butyl lithium (2 equivalents), in a similar manner to Description 28. 1,1-Dimethylethyl 4-{5-[(2-cyanophenyl)sulfonyl]-2,3-dihydro-1H-indol-1-yl}-1-piperidinecarboxylate (D37) was purified by silica gel chromatography, then using mass directed preparative HPLC, and was obtained as a yellow oil (14%).

MS: m/z (M-^{t}Bu+H)⁺ 412, C₂₅H₂₉N₃O₄S requires 467.

### DESCRIPTION 38

### 1,1-Dimethylethyl 4-{5-[(3-cyanophenyl)sulfonyl]-2,3-dihydro-1H-indol-1-yl}-1-piperidinecarboxylate (D38)

Prepared from 1,1-dimethylethyl 4-(5-iodo-2,3-dihydro-1H-indol-1-yl)-1-piperidinecarboxylate (D6) and 3-cyanobenzenesulfonyl fluoride (D17) using t-butyl lithium (2 equivalents), in a similar manner to Description 28. 1,1-Dimethylethyl 4-{5-[(3-cyanophenyl)sulfonyl]-2,3-dihydro-1H-indol-1-yl}-1-piperidinecarboxylate (D38) was purified on silica and then using mass-directed preparative HPLC and was obtained as a yellow oil (36%).

MS: m/z (M-^{t}Bu+H)⁺ 412, C₂₅H₂₉N₃O₄S requires 467.

### DESCRIPTION 39

### 1,1-Dimethylethyl 4-{5-[(2-fluorophenyl)sulfonyl]-1H-indol-1-yl}-1-piperidinecarboxylate (D39)

Prepared from 1,1-dimethylethyl 4-{5-[(2-fluorophenyl)sulfonyl]-2,3-dihydro-1H-indol-1-yl}-1-piperidinecarboxylate (D46) using 2,3-dichloro-5,6-dicyano-1,4-benzoquinone (1.1 equivalents) in a similar manner to Description 2. An additional 0.5 equivalents of 2,3-dichloro-5,6-dicyano-1,4-benzoquinone was added during the reaction and the reaction stirred for a total of 18 h. 1,1-Dimethylethyl 4-{5-[((2-fluorophenyl)sulfonyl]-1H-indol-1-yl}-1-piperidinecarboxylate (D39) was obtained as a yellow oil (95%).

MS: m/z (M-^{t}Bu+H)⁺ 403, C₂₄H₂₇FN₂O₄S requires 458.

### DESCRIPTION 40

### 1,1-Dimethylethyl 4-{5-[(3-fluorophenyl)sulfonyl]-1H-indol-1-yl}-1-piperidinecarboxylate (D40)

Prepared from 1,1-dimethylethyl 4-{5-[(3-fluorophenyl)sulfonyl]-2,3-dihydro-1H-indol-1-yl}-1-piperidinecarboxylate (D45) using 2,3-dichloro-5,6-dicyano-1,4-benzoquinone (1.5 equivalents) in a similar manner to Description 2. The reaction was stirred for a total of 18 h. 1,1-Dimethylethyl 4-{5-[(3-fluorophenyl)sulfonyl]-1H-indol-1-yl}-1-piperidinecarboxylate (D40) was obtained as a yellow solid (62%).

MS: m/z (M-^{t}Bu+H)⁺ 403, C₂₄H₂₇FN₂O₄S requires 458.

### DESCRIPTION 41

### 1,1-Dimethylethyl 4-(5-{[3-(methyloxy)phenyl]sulfonyl}-2,3-dihydro-1H-indol-1-yl)-1-piperidinecarboxylate (D41)

In an oven dried 3-necked round bottomed flask 1,1-dimethylethyl 4-(5-bromo-2,3-dihydro-1H-indol-1-yl)-1-piperidinecarboxylate (D1) (400 mg, 1.05 mmol) was dissolved in dry THF (5 ml) and cooled to −78° C. under argon atmosphere. tert-Butyllithium (1.5M in pentane, 1.4 ml, 2.1 mmol) was added dropwise and the mixture was stirred for 10 minutes before a solution of 3-(Methyloxy)benzenesulfonyl fluoride (D25) (299 mg, 1.57 mmol) in dry THF (2 ml) was added. The reaction mixture was kept at −78° C. before being allowed to warm to room temperature. The reaction was monitored by LC-MS. After 1 hour the reaction was quenched with water and extracted with diethyl ether; the organics were washed with brine, dried over MgSO₄ and concentrated to afford a yellow oil (550 mg). The crude was purified by flash chromatography (20 g silica column) with a gradient of EtOAc in hexane to produce the desired product 1,1-Dimethylethyl 4-(5-{[3-(methyloxy)phenyl]sulfonyl}-2,3-dihydro-1H-indol-1-yl)-1-piperidinecarboxylate (D41) as a colourless oil (270 mg, 45%).

¹H-NMR (CDCl₃): δ 1.46 (9H, s), 1.60 (2H, m), 1.75 (2H, m), 2.77 (2H, bt), 3.00 (2H, t), 3.51 (3H, m), 3.83 (3H, s), 4.25 (2H, bd), 6.32 (1H, d), 7.00 (1H, dd), 7.45 (4H, m), 7.65 (1H, dd).

### DESCRIPTION 42

### 1,1-Dimethylethyl 4-{5-[(2,4-dimethyl-1,3-thiazol-5-yl)sulfonyl]-2,3-dihydro-1H-indol-1-yl}-1-piperidinecarboxylate (D42)

Prepared using a method analogous to Description 41 but using the sulfonyl fluoride (D24).

MS: m/z (M+Na⁺)+500, C₂₃H₃₁N₃O₄S₂ requires 477.

### DESCRIPTION 43

### 1,1-Dimethylethyl 4-{5-[(1,3,5-trimethyl-1H-pyrazol-4-yl)sulfonyl]-2,3-dihydro-1H-indol-1-yl}-1-piperidinecarboxylate (D43)

Prepared using a method analogous to Description 41 but using the sulfonyl fluoride (D23).

MS: m/z (M+Na⁺)+497, C₂₄H₃₄N₄O₄S requires 474.

### DESCRIPTION 44

### 1,1-Dimethylethyl 4-{5-[(2,5-difluorophenyl)sulfonyl]-2,3-dihydro-1H-indol-1-yl}-1-piperidinecarboxylate (D44)

1,1-Dimethylethyl 4-(5-iodo-2,3-dihydro-1H-indol-1-yl)-1-piperidinecarboxylate (D6) (1.5 g, 3.5 mmol) in dry THF (15 ml) was cooled to −78° C. under argon and tert-BuLi (1.5M soln. in pentane; 4.7 ml, 7 mmol) was added dropwise. The resulting pale yellow solution was stirred at −78° C. for 10 mins and then a solution of 2,5-difluorobenzenesulfonyl fluoride (D19) (1.03 g, 5.25 mmol) in dry THF (2 ml) was added dropwise. The reaction solution was stirred at −78° C. for 0.5 h and then allowed to warm to RT over 1 h. Satd. aq. NH₄Cl soln. was added and the reaction was diluted with EtOAc. It was then washed with brine, dried (MgSO₄), filtered and evaporated to leave the crude product (1.9 g). This was purified on silica eluting with a gradient of 0-40% EtOAc in hexane to afford 1,1-dimethylethyl 4-{5-[(2,5-difluorophenyl)sulfonyl]-2,3-dihydro-1H-indol-1-yl}-1-piperidinecarboxylate (D44) (1 g, 60%).

¹H-NMR (CDCl₃): δ 1.47 (9H, s), 1.58 (2H, m), 1.75 (2H, m), 2.77 (2H, bt), 3.02 (2H, t), 3.54 (3H), 4.12 (2H, bs), 6.35 (1H, d), 7.06 (1H, m), 7.18 (1H, m), 7.53 (1H, s), 7.75 (2H, m)

### DESCRIPTION 45

### 1,1-Dimethylethyl 4-{5-[(3-fluorophenyl)sulfonyl]-2,3-dihydro-1H-indol-1-yl}-1-piperidinecarboxylate (D45)

1,1-Dimethylethyl 4-(5-iodo-2,3-dihydro-1H-indol-1-yl)-1-piperidinecarboxylate (D6) (856 mg, 2.0 mmol) in dry THF (10 ml) was cooled to −78° C. under argon and n-BuLi (2.5M soln. in hexanes; 0.880 ml, 2.2 mmol) was added dropwise. The resulting pale yellow solution was stirred at −78° C. for 15 mins and the a solution of 3-fluorobenzenesulfonyl fluoride (D15) (534 mg, 3.0 mmol) in dry THF (1 ml) was added dropwise. The reaction solution was stirred at −78° C. for 0.5 h and then allowed to warm to RT over 1 h. Satd. NH₄Cl soln. (2 ml) was added and the reaction was diluted with EtOAc. It was then washed with brine, dried (MgSO₄), filtered and evaporated to leave the crude product. This was purified on silica eluting with hexane/EtOAc (4:1 to 2:1) to afford 1,1-Dimethylethyl 4-{5-[((3-fluorophenyl)sulfonyl]-2,3-dihydro-1H-indol-1-yl}-1-piperidinecarboxylate (D45) (214 mg, 23%).

¹H NMR (CDCl₃) δ 1.47 (9H, s), 1.55 (2H, m), 1.74 (2H, m), 2.77 (2H, m), 3.00 (2H, t J=8.6 Hz), 3.52 (3H, m), 4.24 (2H, br.m), 6.34 (1H, d J=8.4 Hz) 7.19 (1H, m), 7.43 (2H, m), 7.57 (1H, m) and 7.68 (2H, m). MS (electrospray): m/z (M+H)⁺ 461; C₂₄H₂₉FN₂O₄S requires M=460.

### DESCRIPTION D46

### 1,1-Dimethylethyl 4-{5-[(2-fluorophenyl)sulfonyl]-2,3-dihydro-1H-indol-1-yl}-1-piperidinecarboxylate (D46)

1,1-Dimethylethyl 4-(5-iodo-2,3-dihydro-1H-indol-1-yl)-1-piperidinecarboxylate (D6) (656 mg, 1.53 mmol) in dry THF (8 ml) was cooled to −78° C. under argon and sec-BuLi (1.4M soln. in cyclohexane; 1.31 ml, 1.84 mmol) was added dropwise. The resulting pale yellow solution was stirred at −78° C. for 10 mins and then a solution of 2-fluorobenzenesulfonyl fluoride (D48) (422 mg, 2.37 mmol) in dry THF (2 ml) was added dropwise. The reaction solution was stirred at −78° C. for 1 h and then allowed to warm to RT over 1 h. Satd. NH₄Cl soln. (2 ml) was added and the reaction was diluted with EtOAc. It was then washed with brine, dried (MgSO₄), filtered and evaporated to leave the crude product 1,1-dimethylethyl 4-{5-[(2-fluorophenyl)sulfonyl]-2,3-dihydro-1H-indol-1-yl}-1-piperidinecarboxylate (D46). This was purified on silica eluting with hexane/EtOAc (4:1 to 2:1) to afford the title compound (257 mg, 37%).

¹H NMR (CDCl₃) δ 1.47 (9H, s), 1.55 (2H, m), 1.75 (2H, m), 2.77 (2H, m), 3.00 (2H, t J=8.6 Hz), 3.52 (3H, m), 4.24 (2H, br.m), 6.35 (1H, d J=8.4 Hz) 7.06 (1H, m), 7.26 (1H, m, partially obscured by CHCl₃), 7.52 (2H, m), 7.72 (1H, m) and 8.04 (1H, m). MS (electrospray): m/z (M+H)⁺ 461; C₂₄H₂₉FN₂O₄S requires M=460.

### DESCRIPTION 47

### 1,1-Dimethylethyl 4-{5-[(3,5-difluorophenyl)sulfonyl]-2,3-dihydro-1H-indol-1-yl}-1-piperidinecarboxylate (D47)

1,1-Dimethylethyl 4-(5-iodo-2,3-dihydro-1H-indol-1-yl)-1-piperidinecarboxylate (D6) (642 mg, 1.5 mmol) in dry THF (8 ml) was cooled to −78° C. under argon and tert-BuLi (1.5M soln. in pentane; 2.0 ml, 3.0 mmol) was added dropwise. The resulting pale yellow solution was stirred at −78° C. for 10 mins and then a solution of 3,5-difluorobenzenesulfonyl fluoride (D52) (441 mg, 2.25 mmol) in dry THF (2 ml) was added dropwise. The reaction solution was stirred at −78° C. for 1.25 h and then allowed to warm to RT over 2.5 h. Satd. NH₄Cl soln. (2 ml) was added and the reaction was diluted with EtOAc. It was then washed with brine, dried (MgSO₄), filtered and evaporated to leave the crude product. This was purified on silica eluting with a gradient of 0-25% EtOAc in hexane to afford 1,1-dimethylethyl 4-{5-[(3,5-difluorophenyl)sulfonyl]-2,3-dihydro-1H-indol-1-yl}-1-piperidinecarboxylate (D47) (216 mg, 30%).

¹H NMR (CDCl₃) δ 1.47 (9H, s), 1.55 (2H, m), 1.75 (2H, m), 2.77 (2H, m), 3.02 (2H, t J=8.6 Hz), 3.54 (3H, m), 4.25 (2H, br.m), 6.35 (1H, d J=8.4 Hz), 6.92 (1H, m), 7.40 (2H, m), 7.45 (1H, d J=1.6 Hz) and 7.64 (1H, m). MS (electrospray): m/z (M+H)⁺ 479; C₂₄H₂₈F₂N₂O₄S requires M=478.

### DESCRIPTION 48

### 2-Fluorobenzenesulfonyl fluoride (D48)

A 4:1 mixture of calcium fluoride and potassium fluoride (2.91 g, loading 3.45 mmol/g), which had been prepared in a similar manner to “J. C. S. Chem. Comm., (1986), 793”, was added to a solution of 2-fluorobenzenesulfonyl chloride (0.066 ml, 0.973 g, 5.0 mmol) in acetonitrile (10 ml), and the mixture stirred at room temperature for 48 h. The calcium fluoride and potassium fluoride were removed by filtering through celite, washing with additional acetonitrile (2×5 ml). The resulting solution was evaporated to dryness. 2-Fluorobenzenesulfonyl fluoride (D48) was obtained as a colourless oil (470 mg, 53%).

¹³C-NMR (CDCl₃): δ 118.5 (d, J=20 Hz), 122.3 (m), 125.7 (s), 131.6 (s), 138.9 (s), 160.5 (d, J=260 Hz).

### DESCRIPTION 49

### 2-Chlorobenzenesulfonyl fluoride (D49)

See Table 1.

### DESCRIPTION 50

### 3-Chlorobenzenesulfonyl fluoride (D50)

See Table 1.

### DESCRIPTION 51

### 4-chlorobenzenesulfonyl fluoride (D51)

See Table 1.

### DESCRIPTION 52

### 3,5-Difluorobenzenesulfonyl fluoride (D52)

See Table 1.

The following Descriptions were made using the appropriate sulfonyl chloride and a method analogous to that specified for Description 15 or 48.

TABLE 1

| | | Method | | |
|---|---|---|---|---|
| Description | | similar to that | | |
| number | Sulfonyl chloride | described in: | Characterisation | Notes |
| 16 | 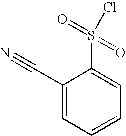 | D15 | MS: m/z (M + H)⁺ 186,C₇H₄FNO₂S requires 185. | 91% |
| 17 | 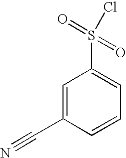 | D15 | MS: m/z (M + H)⁺ 186,C₇H₄FNO₂S requires 185. | 89% |
| 18 | 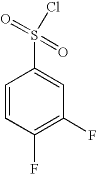 | D15 | ¹³C-NMR (CDCl₃): δ 118.7(d, J = 20 Hz), 119.2 (d,J = 10 Hz), 126.2 (s), 129.5(m), 147.9 (dd, J = 260 Hz& 10 Hz), 152.6 (dd,J = 260 Hz & 10 Hz). | Colourlessoil. 84% |
| 19 | 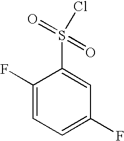 | D15 | ¹³C-NMR (CDCl₃): δ 117.6(dd, J = 20 Hz & 5 Hz),119.5 (dd, J = 20 Hz,10 Hz), 122.5 (m), 124.9(dd, J = 20 Hz & 10 Hz),155.7 (d, J = 250 Hz), 157.7(J = 240 Hz). | White solid89% |
| 20 | 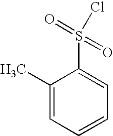 | D15 | ¹³C-NMR (CDCl₃): δ 21.9(s), 128.5 (2C, s), 130.2(m), 130.3 (20, s), 147.1(s). | White solid92% |
| 21 | 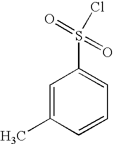 | D15 | ¹³C-NMR (CDCl₃): δ 21.3(s), 125.6 (s), 128.6 (s),129.5 (s), 132.9 (d,J = 20 Hz), 136.4 (s), 140.2(s). | Colourlessoil 94% |
| 22 | 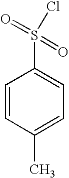 | D15 | ¹³C-NMR (CDCl₃): δ 20.3(s), 126.6 (s), 130.1 (s),132.3 (d, J = 20 Hz), 132.8(s), 135.3 (s), 139.0 (s). | Colourlessoil 92% |
| 23 | 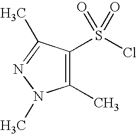 | D15 | MS: m/z (M + H⁺)⁺ 193,C₆H₉FN₂O₂S requires 192 | |
| 24 | 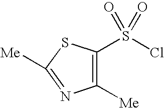 | D15 | MS: m/z (M + H⁺)⁺ 196,C₅H₆FNO₂S₂ requires195. | |
| 25 | 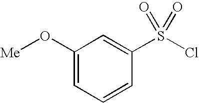 | D15 | ¹H-NMR (CDCl₃): δ 3.90(3H, s), 7.27 (1H, d), 7.47(1H, s), 7.51 (1H, t), 7.60(1H, d)¹⁹F-NMR (CDCl₃): δ-194.8 s | |
| 49 | 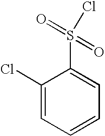 | D48 | ¹³C-NMR (CDCl₃): δ 127.4(s), 131.8 (s), 132.0 (d,J = 24 Hz), 132.4 (s), 133.6(s), 136.2 (s). | Yellowcrystallinesolid 96% |
| 50 | 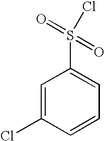 | D48 | ¹³C-NMR (CDCl₃): δ 126.5(s), 128.5 (s), 131.0 (s),134.6 (d, J = 20 Hz), 135.8(s), 136.0 (s). | White oil89% |
| 51 | 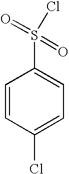 | D48 | ¹³C-NMR (CDCl₃): δ 129.9(2C, s), 130.1 (2C, s),131.4 (d, J = 20 Hz), 142.7(s). | White solid89% |
| 52 | 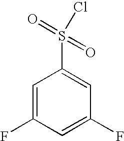 | D15 | ¹³C-NMR (CDCl₃): δ 111.5(t, J = 25 Hz), 112.2-112.5(2C, m), 135.7 (m),162.9 (m). | Yellow oil |

### DESCRIPTION 53

### 1,1-Dimethylethyl 4-[3-[(dimethylamino)methyl]-5-(phenylsulfonyl)-1H-indol-1-yl]-1-piperidinecarboxylate (D53)

1,1-Dimethylethyl 4-[5-(phenylsulfonyl)-1H-indol-1-yl]-1-piperidinecarboxylate (D3) (414 mg, 0.943 mmol) and Eschenmoser's salt (272 mg, 1.47 mmol) were stirred in DMF at 55° C. under argon for 1 h. After cooling, the reaction solution was evaporated to dryness and the residue was partitioned between EtOAc and satd. NaHCO₃ solution. The organic layer was separated and washed with further satd. NaHCO₃ solution (×1) and brine (×1). It was then dried over MgSO₄, filtered and evaporated to afford a beige foam (495 mg). This was purified on silica (20 g) eluting with a gradient of 0-5% MeOH in DCM to afford 1,1-dimethylethyl 4-[3-[(dimethylamino)methyl]-5-(phenylsulfonyl)-1H-indol-1-yl]-1-piperidinecarboxylate (D53) (337 mg, 90%).

¹H NMR (CDCl₃) δ 1.49 (9H, s), 1.89 (2H, m), 2.02 (2H, m), 2.28 (6H, s), 2.90 (2H, m), 3.63 (2H, s), 4.34 (3H, m), 7.26 (1H, s, obscured by CHCl₃), 7.40 (1H, d J=8.8 Hz), 7.50 (3H, m), 7.72 (1H, d J=8.8 Hz), 7.97 (2H, m) and 8.38 (1H, s). MS (electrospray): m/z (M+H)⁺ 498; C₂₇H₃₅N₃O₄S requires M=497.

### DESCRIPTION 54

### 1,1-Dimethylethyl 4-[3-methyl-5-(phenylsulfonyl)-1H-indol-1-yl]-1-piperidinecarboxylate (D54)

1,1-Dimethylethyl 4-[3-[(dimethylamino)methyl]-5-(phenylsulfonyl)-1H-indol-1-yl]-1-piperidinecarboxylate (D53) (337 mg, 0.678 mmol) in ethanol (15 ml) was hydrogenated over 10% Pd/C (200 mg) at room temperature and pressure for 19 h. LC/MS showed little reaction after this time. The catalyst was filtered and the filtrate was hydrogenated over 10% Pd/C (500 mg) at 60 psi for 56 h; LC/MS showed about 30% conversion. The catalyst was filtered and the filtrate evaporated to leave a colourless oil (278 mg). This was purified on silica (10 g) eluting with hexane/EtOAc (3:1 to 3:2) to afford 1,1-dimethylethyl 4-[3-methyl-5-(phenylsulfonyl)-1H-indol-1-yl]-1-piperidinecarboxylate (D54) as a colourless oil (80 mg).

¹H NMR (CDCl₃) δ 1.49 (9H, s), 1.87 (2H, m), 2.00 (2H, m), 2.34 (3H, s), 2.89 (2H, m), 4.31 (3H, m), 7.06 (1H, s), 7.37 (1H, d J=8.8 Hz), 7.49 (3H, m), 7.72 (1H, d J=8.8 Hz), 7.96 (2H, m) and 8.25 (1H, s). MS (electrospray): m/z (M+Na)+477; C₂₅H₃₀N₂O₄S requires M=454.

### EXAMPLE 1

### 5-(Phenylsulfonyl)-1-(4-piperidinyl)-1H-indole hydrochloride (E1)

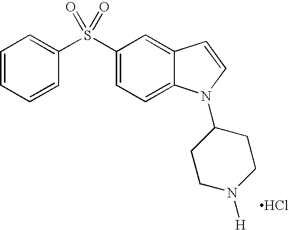

1,1-Dimethylethyl 4-[5-(phenylsulfonyl)-1H-indol-1-yl]-1-piperidinecarboxylate (D3) (290 mg, 0.65 mmol) was dissolved in 4M HCl in 1,4-dioxane (20 ml) at room temperature. After 1 h, the solvent was evaporated to give 5-(phenylsulfonyl)-1-(4-piperidinyl)-1H-indole hydrochloride (E1) as a white solid (245 mg):

¹H NMR (CD₃OD) [symbol] 2.21-2.28 (4H, m), 3.26-3.34 (2H, m), 3.54 (2H, d), 4.81 (1H, m), 6.74 (1H, d) 7.50-7.60 (4H, m), 7.71 (2H, s), 7.93 (2H, d), 8.27 (1H, s); MS: m/z (M+H)⁺ 341; C₁₉H₂OSN₂O₂ requires: 340.

### EXAMPLE 2

### 1-(1-Methyl-4-piperidinyl)-5-(phenylsulfonyl)-1H-indole hydrochloride (E2)

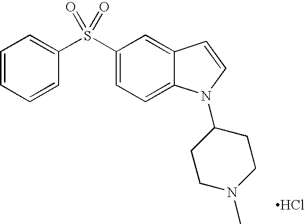

A mixture of 5-(phenylsulfonyl)-1-(4-piperidinyl)-1H-indole hydrochloride (E1) (120 mg, 0.32 mmol), 37% formaldehyde in water (0.2 ml), and sodium triacetoxyborohydride (0.26 g, 1.3 mmol), was suspended in 1,2-dichloroethane (5 ml) and stirred for 18 hours at room temperature. The mixture was filtered through an SCX cartridge (10 g) washing with dichloromethane (2×10 ml) and methanol (2×10 ml) then the product eluted with 10% aqueous ammonia (d=0.88) in methanol, to give the 1-(1-methyl-4-piperidinyl)-5-(phenylsulfonyl)-1H-indole as a white solid. This was treated with 1M HCl in Et₂O (1 ml) and evaporated to give 1-(1-methyl-4-piperidinyl)-5-(phenylsulfonyl)-1H-indole hydrochloride (E2) (60 mg):

¹H NMR (d₆-DMSO) [symbol] 2.15 (2H, d), 2.36 (2H, dd), 2.80 (3H, d), 3.18 (2H, dd), 3.55 (2H, d), 4.77 (1H, brt.), 6.75 (1H, d), 7.55-7.65 (4H, m), 7.70 (1H, dd), 7.82 (1H, d), 7.94 (1H, d), 8.27 (2H, d), 10.4 (1H, br.s); MS: m/z (M+H)⁺ 355; C₂₀H₂₂SN₂O₂ requires: 354.

### EXAMPLE 3

### 5-(Phenylsulfonyl)-1-(4-piperidinyl)-2,3-dihydro-1H-indole hydrochloride (E3a)

### Case (i)

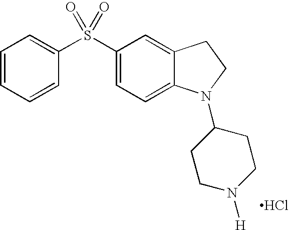

1,1-Dimethylethyl 4-[5-(phenylsulfonyl)-2,3-dihydro-1H-indol-1-yl]-1-piperidinecarboxylate (D4), (234 mg, 0.52 mmol) was dissolved in 4M HCl in 1,4-dioxane (20 ml) at room temperature. After 1 h, the solvent was evaporated to give 5-(phenylsulfonyl)-1-(4-piperidinyl)-2,3-dihydro-1H-indole hydrochloride (E3a) as a white solid (200 mg):

¹H NMR (CD₃OD) [symbol] 1.86-2.01 (4H, m), 3.01 (2H, t), 3.15 (2H, dt), 3.48-3.67 (4H, m), 3.89 (1H, tt), 6.59 (1H, d) 7.49-7.63 (5H, m), 7.86 (2H, d); MS: m/z (M+H)⁺ 343; C₁₉H₂₂SN₂O₂ requires: 342.

### Case (ii)

Compound D4 (6.5 g, 14.7 mmol) was dissolved in MeOH (200 ml) and HCl (4M in 1,4-dioxane, 100 ml) was added at once to give a purple coloured solution which was stirred at room temperature for 15 minutes. The reaction mixture was then concentrated to dryness and the resulting material was recrystallised from EtOH (130 ml) to afford the desired product (E3a) (4.54 g, 82%).

### 5-(Phenylsulfonyl)-1-(4-piperidinyl)-2,3-dihydro-1H-indole (E3b)

### Case (i)

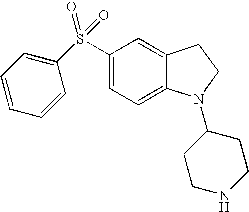

5-(Phenylsulfonyl)-1-(4-piperidinyl)-2,3-dihydro-1H-indole hydrochloride (E3a) (90 mg) was dissolved in ethyl acetate (25 ml) and washed with sodium bicarbonate, then brine, dried with magnesium sulphate filtered and evaporated to dryness. 5-(Phenylsulfonyl)-1-(4-piperidinyl)-2,3-dihydro-1H-indole (E3b) was obtained as a yellow oil (60 mg).

MS: m/z (M+H)⁺ 343, C₁₉H₂₂N₂O₂S requires 342.

### Case (ii)

1,1-Dimethylethyl 4-[5-(phenylsulfonyl)-2,3-dihydro-1H-indol-1-yl]-1-piperidine-carboxylate (D4) (1.39 g) was stirred in 4M HCl in 1,4-dioxane (5 ml) at room temperature for 1 h. The solvent was evaporated and the resulting white solid was partitioned between ethyl acetate (250 ml) and water (250 ml). Two drops of concentrated aq. sodium hydroxide were added to make the solution basic. The organic phase was separated and the aqueous layer extracted with ethyl acetate (250 ml). The organic phases were combined and washed with brine (100 ml), dried with magnesium sulphate, filtered and evaporated to dryness, producing 1.02 g of yellow gum. The gum was recrystallised from ethyl acetate, producing 130 mg of white solid. The residues were evaporated to dryness, producing 890 mg of off white foam. 5-(Phenylsulfonyl)-1-(4-piperidinyl)-2,3-dihydro-1H-indole (E3b) was obtained (130 mg+890 mg), consistent spectroscopically with the material produced in Case (i).

### EXAMPLE 4

### 1-(1-Methyl-4-piperidinyl)-5-(phenylsulfonyl)-2,3-dihydro-1H-indole hydrochloride (E4)

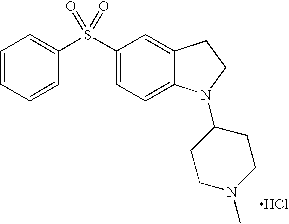

A mixture of 5-(phenylsulfonyl)-1-(4-piperidinyl)-2,3-dihydro-1H-indole hydrochloride (E3a) (170 mg, 0.45 mmol), 37% formaldehyde in water (0.2 ml), and sodium triacetoxyborohydride (0.382 mg, 1.8 mmol), was suspended in 1,2-dichloroethane (6 ml) and stirred for 18 hours at room temperature. The mixture was filtered through an SCX cartridge (10 g) washing with dichloromethane (2×15 ml) and methanol (2×15 ml) then the product eluted with 10% aqueous ammonia (d=0.88) in methanol, to give a white solid. This was treated with 1M HCl in Et₂O (1 ml) and evaporated to give 1-(1-methyl-4-piperidinyl)-5-(phenylsulfonyl)-2,3-dihydro-1H-indole hydrochloride (E4) (137 mg):

¹H NMR (d₆-DMSO) δ 1.82 (2H, d), 2.00 (2H, dd), 2.71 (3H, d), 2.97 (2H, t), 3.05 (2H, dd), 3.40 (4H, dd), 3.79 (1H, tt), 6.59 (1H, d), 7.46 (1H, s), 7.54-7.63 (4H, m), 7.86 (2H, dd), 10.5 (1H, br.s); MS: m/z (M+H)⁺ 357 C₂₀H₂₄SN₂O₂ requires: 356.

### EXAMPLE 5

### 3,3-Dimethyl-1-(1-methyl-4-piperidinyl)-5-(phenylsulfonyl)-2,3-dihydro-1H-indole hydrochloride (E5)

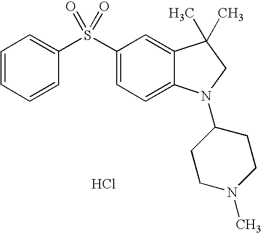

To a suspension of 5-Iodo-3,3-dimethyl-1-(1-methyl-4-piperidinyl)-2,3-dihydro-1H-indole (D8) (370 mg, 1 mmol) and benzenesulfinic acid sodium salt (197 mg, 1.2 mmol) in dry toluene (6 ml) were added Pd₂(dba)₃ (23 mg, 0.025 mmol), 9,9-dimethyl-4,5-bis(diphenylphosphino)xanthene) (xantphos) (29 mg, 0.05 mmol), Cs₂CO₃ (489 mg, 1.5 mmol) and tetrabutylammonium chloride (334 mg, 1.2 mmol). The mixture was stirred under argon at 80° C. and after 15 hrs it was cooled to room temperature, diluted with dichloromethane and filtered through a pad of celite. It was then concentrated to dryness and the crude (800 mg) was applied to a Biotage (25+S) amine cartridge and eluted with a gradient of EtOAc in hexane to isolate 3,3-dimethyl-1-(1-methyl-4-piperidinyl)-5-(phenylsulfonyl)-2,3-dihydro-1H-indole (130 mg, 34%).

¹H-NMR (CDCl₃): δ1.26 (6H, s), 1.75 (4H, m), 2.04 (2H, m), 2.30 (3H, s), 2.94 (2H, d), 3.26 (2H, s), 3.39 (1H, quintet), 6.33 (1H, d, J=8.4 Hz), 7.47 (2H, m), 7.48 (2H, m), 7.62 (1H, d, J=8.4 Hz), 7.89 (2H, d).

The compound was dissolved in methanol and treated with 1 mol eq. of HCl (1M in Et₂O) to make the HCl salt 3,3-Dimethyl-1-(1-methyl-4-piperidinyl)-5-(phenylsulfonyl)-2,3-dihydro-1H-indole hydrochloride (E5) after evaporation.

MS: m/z (M+H)⁺ 385, C₂₂H₂₈N₂O₂S requires 384.

### EXAMPLE 6

### 1-(Octahydro-7-indolizinyl)-5-(phenylsulfonyl)-2,3-dihydro-1H-indole hydrochloride (E6)

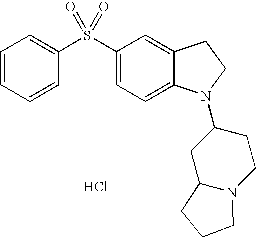

To a solution of 5-bromo-1-(octahydro-7-indolizinyl)-2,3-dihydro-1H-indole (D7) (300 mg, 0.94 mmol) in dry toluene (5.3 ml) were added benzenesulfinic acid sodium salt (230 mg, 1.4 mmol), Pd₂(dba)₃ (22 mg, 0.024 mmol), xantphos (28 mg, 0.05 mmol), Cs₂CO₃ (457 mg, 1.4 mmol) and tetrabutylammonium chloride (312 mg, 1.12 mmol). The mixture was stirred at reflux under an argon atmosphere and after 20 hrs it was cooled to room temperature, filtered through a pad of celite and washed with dichloromethane. It was then concentrated to dryness and the crude (660 mg) was applied to a Biotage (25+M) amine cartridge and eluted with a gradient of EtOAc in hexane. The desired product was further purified by SCX (1 g cartridge, washed with dichloromethane, methanol and eluted with methanolic ammonia, and then using mass-directed preparative HPLC and the product converted to the HCl salt by adding 1 mol eq. of HCl (1M in Et₂O) to a solution of the product in dichloromethane. The compound was triturated with ether affording 1-(octahydro-7-indolizinyl)-5-(phenylsulfonyl)-2,3-dihydro-1H-indole hydrochloride (E6) (11 mg, 3%).

¹H-NMR (CD₃OD): δ 1.78 (2H, m), 2.15 (7H, m), 3.05 (4H, m), 3.60 (3H, m), 3.68 (1H, d), 3.97 (1H, m), 6.58 (1H, d), 7.52 (4H, m), 7.62 (1H, d), 7.86 (2H, d).

MS: m/z (M+H)⁺ 383, C₂₂H₂₆N₂O₂S requires 382.

### EXAMPLE 7

### 1-(4-Methyl-4-piperidinyl)-5-(phenylsulfonyl)-2,3-dihydro-1H-indole hydrochloride (E7)

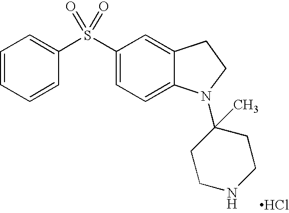

See Table 3

### EXAMPLE 8

### 5-[(3-Fluorophenyl)sulfonyl]-1-(4-piperidinyl)-2,3-dihydro-1H-indole hydrochloride (E8)

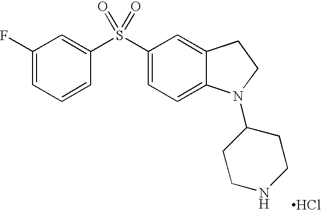

1,1-Dimethylethyl 4-{5-[(3-fluorophenyl)sulfonyl]-2,3-dihydro-1H-indol-1-yl}-1-piperidinecarboxylate (D45) (210 mg, 0.456 mmol) was treated with 4M HCl in dioxane (7.5 ml) and the resulting mixture was stirred at RT for 1.25 h. It was then evaporated to dryness and the resulting white solid was triturated with diethyl ether, filtered and dried in vacuo to afford 5-[(3-fluorophenyl)sulfonyl]-1-(4-piperidinyl)-2,3-dihydro-1H-indole hydrochloride (E8) as an off-white solid (170 mg, 94%).

¹H NMR (d₆-DMSO) δ 1.82 (4H, m), 3.00 (4H, m), 3.35 (2H, m), 3.50 (2H, t J=8.4 Hz), 3.85 (1H, m, partially obscured by water), 6.61 (1H, d, J=8.4 Hz), 7.50 (2H, m), 7.63 (2H, m), 7.71 (2H, m), 8.64 (1H, br, m) and 8.86 (1H, br. m). MS (ES): m/z (M+H)⁺ 361; C₁₉H₂₁FN₂O₂S requires M=360.

### EXAMPLE 9

### 5-[(2-Fluorophenyl)sulfonyl]-1-(4-piperidinyl)-2,3-dihydro-1H-indole hydrochloride (E9)

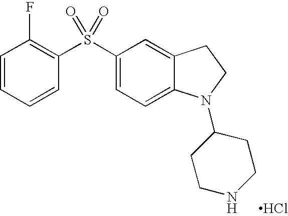

1,1-Dimethylethyl 4-(5-iodo-2,3-dihydro-1H-indol-1-yl)-1-piperidinecarboxylate (D6) (656 mg, 1.53 mmol) in dry THF (8 ml) was cooled to −78° C. under argon and sec-BuLi (1.4M soln. in cyclohexane; 1.31 ml, 1.84 mmol) was added dropwise. The resulting pale yellow solution was stirred at −78° C. for 10 mins and then a solution of 2-fluorobenzenesulfonyl fluoride (D48) (422 mg, 2.37 mmol) in dry THF (2 ml) was added dropwise. The reaction solution was stirred at −78° C. for 1 h and then allowed to warm to RT over 1 h. Satd. NH₄Cl soln. (2 ml) was added and the reaction was diluted with EtOAc. It was then washed with brine, dried (MgSO₄), filtered and evaporated to leave the crude product 1,1-dimethylethyl 4-{5-[(2-fluorophenyl)sulfonyl]-2,3-dihydro-1H-indol-1-yl}-1-piperidinecarboxylate (D46). This was purified on silica eluting with hexane/EtOAc (4:1 to 2:1) to afford the title compound (257 mg, 37%).

¹H NMR (CDCl₃) δ 1.47 (9H, s), 1.55 (2H, m), 1.75 (2H, m), 2.77 (2H, m), 3.00 (2H, t J=8.6 Hz), 3.52 (3H, m), 4.24 (2H, br.m), 6.35 (1H, d J=8.4 Hz) 7.06 (1H, m), 7.26 (1H, m, partially obscured by CHCl₃), 7.52 (2H, m), 7.72 (1H, m) and 8.04 (1H, m). MS (electrospray): m/z (M+H)⁺ 461; C₂₄H₂₉FN₂O₄S requires M=460.

The 1,1-dimethylethyl 4-{5-[(2-fluorophenyl)sulfonyl]-2,3-dihydro-1H-indol-1-yl}-1-piperidinecarboxylate (D46) (250 mg, 0.54 mmol) was treated with 4M HCl in dioxane (7.5 ml) in an analogous manner to that described in E8 to afford 5-[(2-fluorophenyl)sulfonyl]-1-(4-piperidinyl)-2,3-dihydro-1H-indole hydrochloride (E9).

MS (ES): m/z (M+H)⁺ 361; C₁₉H₂₁FN₂O₂S requires M=360.

### EXAMPLE 10

### 5-[(3,5-Difluorophenyl)sulfonyl]-1-(4-piperidinyl)-2,3-dihydro-1H-indole hydrochloride (E10)

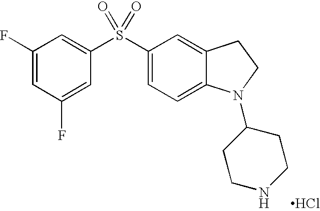

1,1-Dimethylethyl 4-(5-iodo-2,3-dihydro-1H-indol-1-yl)-1-piperidinecarboxylate (D6) (642 mg, 1.5 mmol) in dry THF (8 ml) was cooled to −78° C. under argon and tert-BuLi (1.5M soln. in pentane; 2.0 ml, 3.0 mmol) was added dropwise. The resulting pale yellow solution was stirred at −78° C. for 10 mins and then a solution of 3,5-difluorobenzenesulfonyl fluoride (D52) (441 mg, 2.25 mmol) in dry THF (2 ml) was added dropwise. The reaction solution was stirred at −78° C. for 1.25 h and then allowed to warm to RT over 2.5 h. Satd. NH₄Cl soln. (2 ml) was added and the reaction was diluted with EtOAc. It was then washed with brine, dried (MgSO₄), filtered and evaporated to leave the crude product. This was purified on silica eluting with a gradient of 0-25% EtOAc in hexane to afford 1,1-dimethylethyl 4-{5-[(3,5-difluorophenyl)sulfonyl]-2,3-dihydro-1H-indol-1-yl}-1-piperidinecarboxylate (D47) (216 mg, 30%).

¹H NMR (CDCl₃) δ 1.47 (9H, s), 1.55 (2H, m), 1.75 (2H, m), 2.77 (2H, m), 3.02 (2H, t J=8.6 Hz), 3.54 (3H, m), 4.25 (2H, br.m), 6.35 (1H, d J=8.4 Hz), 6.92 (1H, m), 7.40 (2H, m), 7.45 (1H, d J=1.6 Hz) and 7.64 (1H, m). MS (electrospray): m/z (M+H)⁺ 479; C₂₄H₂₈F₂N₂O₄S requires M=478.

1,1-Dimethylethyl 4-{5-[(3,5-difluorophenyl)sulfonyl]-2,3-dihydro-1H-indol-1-yl}-1-piperidinecarboxylate (D47) was treated with 4M HCl in dioxane in an analogous manner to that described in E8. The product 5-[(3,5-Difluorophenyl)sulfonyl]-1-(4-piperidinyl)-2,3-dihydro-1H-indole hydrochloride (E10) was recrystallised from ethanol.

MS (electrospray): m/z (M+H)⁺ 379; C₁₉H₂₀F₂N₂O₂S requires M=378.

### EXAMPLE 11

### 5-[(4-methylphenyl)sulfonyl]-1-(4-piperidinyl)-2,3-dihydro-1H-indole (E11)

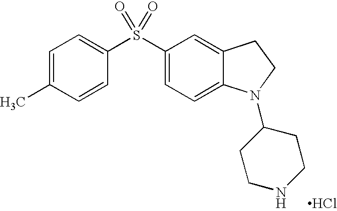

See Table 2.

### EXAMPLE 12

### 5-[(2-Chlorophenyl)sulfonyl]-1-(4-piperidinyl)-2,3-dihydro-1H-indole hydrochloride (E12)

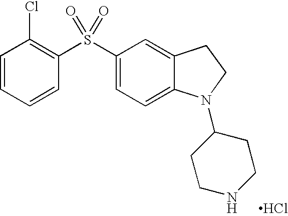

See Table 2.

### EXAMPLE 13

### 5-[(4-Chlorophenyl)sulfonyl]-1-(4-piperidinyl)-2,3-dihydro-1H-indole hydrochloride (E13)

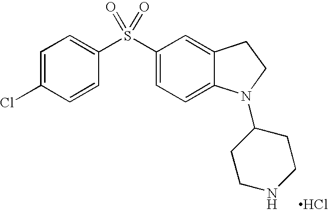

See Table 2.

### EXAMPLE 14

### 5-[(2-fluorophenyl)sulfonyl]-1-(4-piperidinyl)-1H-indole hydrochloride (E14)

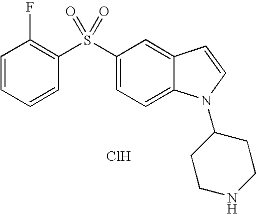

See Table 3

### EXAMPLE 15

### 5-[(4-Fluorophenyl)sulfonyl]-1-(4-piperidinyl)-2,3-dihydro-1H-indole hydrochloride (E15a) and 5-[(4-Fluorophenyl)sulfonyl]-1-(4-piperidinyl)-2,3-dihydro-1H-indole (E15b)

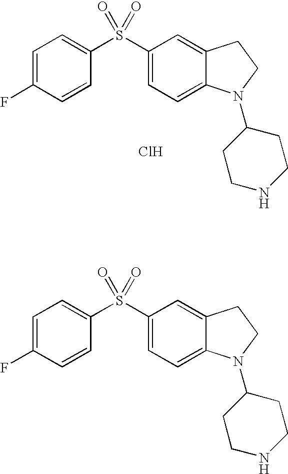

Prepared from 1,1-dimethylethyl 4-{5-[(4-fluorophenyl)sulfonyl]-2,3-dihydro-1H-indol-1-yl}-1-piperidinecarboxylate (D36) in a similar manner to (E3). The crude product was purified using mass-directed preparative HPLC. The appropriate fractions were combined and evaporated to dryness, and then treated with 1M hydrochloric acid in diethyl ether. This was then evaporated to dryness, dissolved in propan-2-ol, filtered, and concentrated. 5-[(4-Fluorophenyl)sulfonyl]-1-(4-piperidinyl)-2,3-dihydro-1H-indole hydrochloride (E15a) was crystallised from hot propan-2-ol to give a white solid (23%).

MS: m/z (M+H)⁺ 361, C₁₉H₂₁FN₂O₂S requires 360. The crystallisation residues were passed down an SCX cartridge eluting with methanol and then methanol ammonia. 5-[(4-Fluorophenyl)sulfonyl]-1-(4-piperidinyl)-2,3-dihydro-1H-indole (E15b) was obtained as a white solid (34%).

¹H-NMR, (CDCl₃): δ 1.61 (2H, m), 1.76 (2H, m), 2.69 (2H, td, J=12.2 Hz & 2.4 Hz), 2.99 (2H, t, J=8.6 Hz), 3.19 (2H, m), 3.47 (1H, m), 3.56 (2H, t, J=8.6 Hz), 6.32 (1H, d, J=8.4 Hz), 7.12 (2H, m), 7.44 (1H, d, J=1.6 Hz), 7.62 (1H, dd, J=8.4 Hz & 2.0 Hz), 7.89 (2H, m).

### EXAMPLE 16

### 1-(Hexahydro-1H-azepin-4-yl)-5-(phenylsulfonyl)-2,3-dihydro-1H-indole (E16a) and 1-(Hexahydro-1H-azepin-4-yl)-5-(phenylsulfonyl)-2,3-dihydro-1H-indole

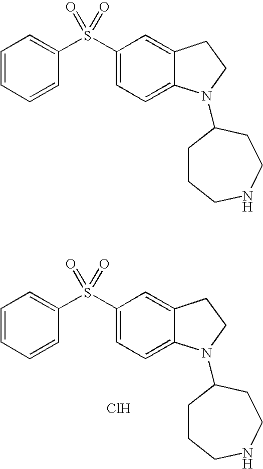

Prepared from 1,1-dimethylethyl 4-[5-(phenylsulfonyl)-2,3-dihydro-1H-indol-1-yl]hexahydro-1H-azepine-1-carboxylate (D33) in a similar manner to the method of Example (E3a). In this case, the product was then purified using an SCX cartridge eluting with methanol and then methanol ammonia. 1-(Hexahydro-1H-azepin-4-yl)-5-(phenylsulfonyl)-2,3-dihydro-1H-indole (E16a) was obtained as a white solid (99%).

MS: m/z (M+H)⁺ 357, C₂₀H₂₄N₂O₂S requires 356.

A sample of 1-(hexahydro-1H-azepin-4-yl)-5-(phenylsulfonyl)-2,3-dihydro-1H-indole (E16a) was then treated with 1M hydrochloric acid in diethyl ether, evaporated to dryness and freeze dried from water. 1-(Hexahydro-1H-azepin-4-yl)-5-(phenylsulfonyl)-2,3-dihydro-1H-indole hydrochloride was obtained as a white solid (E16b).

¹H-NMR (CDCl₃): δ 1.93-2.23 (6H, m), 3.01 (2H, t, J=8.2 Hz), 3.17 (2H, m) 3.36-3.49 (2H, m), 3.58 (2H, t, J=8.4 Hz), 3.84 (1H, m), 6.53 (1H, d, J=8.4 Hz), 7.45-7.53 (4H, m), 7.69 (1H, d, J=8.0 Hz), 7.88 (2H, m), 9.79 (2H, s broad). MS: m/z (M+H)⁺ 357, C₂₀H₂₄N₂O₂S requires 356.

### EXAMPLE 17

### 5-(Phenylsulfonyl)-1-(3-pyrrolidinyl)-2,3-dihydro-1H-indole (E17a) and 5-(Phenylsulfonyl)-1-(3-pyrrolidinyl)-2,3-dihydro-1H-indole hydrochloride (E17b)

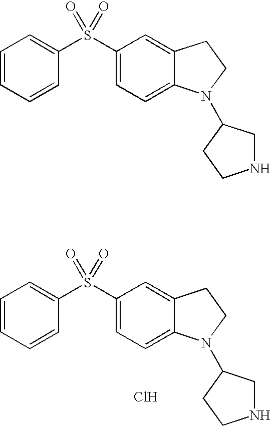

Prepared from 1,1-dimethylethyl 3-[5-(phenylsulfonyl)-2,3-dihydro-1H-indol-1-yl]-1-pyrrolidinecarboxylate (D32) in a similar manner to the method of Example 3a. The product was purified using an SCX cartridge eluting with methanol and then methanolic ammonia. 5-(Phenylsulfonyl)-1-(3-pyrrolidinyl)-2,3-dihydro-1H-indole (E17a) was obtained as a white solid (87%).

MS: m/z (M+H)⁺ 329, C₁₈H₂₀N₂O₂S requires 328. A sample of 5-(Phenylsulfonyl)-1-(3-pyrrolidinyl)-2,3-dihydro-1H-indole (E17a) was then treated with 1M hydrochloric acid in diethyl ether, evaporated to dryness and freeze dried from water. 5-(Phenylsulfonyl)-1-(3-pyrrolidinyl)-2,3-dihydro-1H-indole hydrochloride (E17b) was obtained as a white solid.

¹H-NMR (CDCl₃): δ 2.21 (2H, m), 3.04 (2H, t, J=8.2 Hz), 3.28-3.61 (6H, m), 4.42 (1H, m), 6.43 (1H, d, J=8.0 Hz), 7.46-7.54 (4H, m), 7.80 (1H, d, J=7.6 Hz), 7.89 (2H, d, J=8.4 Hz) 10.02 (2H, s broad). MS: m/z (M+H)⁺ 329, C₁₈H₂₀N₂O₂S requires 328.

### EXAMPLE 18

### 7-Fluoro-5-(phenylsulfonyl)-1-(4-piperidinyl)-2,3-dihydro-1H-indole hydrochloride (E18a) and 7-fluoro-5-(phenylsulfonyl)-1-(4-piperidinyl)-2,3-dihydro-1H-indole (E18b)

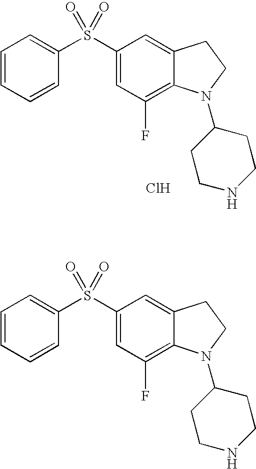

Prepared from 1,1-dimethylethyl 4-[7-fluoro-5-(phenylsulfonyl)-2,3-dihydro-1H-indol-1-yl]-1-piperidinecarboxylate (D34) in a similar manner to the method of (Example 3a). 7-Fluoro-5-(phenylsulfonyl)-1-(4-piperidinyl)-2,3-dihydro-1H-indole hydrochloride (E18a) was crystallised from ethanol and a little diethyl ether giving a white solid (39%).

¹H-NMR (CDCl₃): δ 1.98 (2H, m), 2.19 (2H, m), 2.96 (2H, m), 3.03 (2H, t, J=8.6 Hz), 3.54 (4H, m), 4.11 (1H, m), 7.40 (1H, d, J=1.6 Hz), 7.45 (1H, m), 7.49-7.56 (3H, m), 7.89 (2H, m), 9.59 (1H, broad s), 9.74 (1H, broad s). MS: m/z (M+H)⁺ 361, C₁₉H₂₁ClN₂O₂S requires 360.

The crystallisation residues were passed down an SCX cartridge eluting with methanol and then methanol ammonia. 7-Fluoro-5-(phenylsulfonyl)-1-(4-piperidinyl)-2,3-dihydro-1H-indole (E18b) was obtained as a colourless oil (41%).

¹H-NMR (CDCl₃): δ 1.64 (2H, m), 1.75 (2H, m), 2.69 (2H, td, J=12.2 Hz & 2.4 Hz), 3.01 (2H, t, J=8.8 Hz), 3.18 (2H, m), 3.56 (2H, t, J=9.0 Hz), 3.94 (1H, m), 7.32 (1H, d, J=1.2 Hz), 7.39 (1H, dd, J=12.2 Hz & 1.8 Hz), 7.46-7.55 (3H, m), 7.90 (2H, m).

### EXAMPLE 19

### 7-Chloro-5-(phenylsulfonyl)-1-(4-piperidinyl)-2,3-dihydro-1H-indole hydrochloride (E19a) and 7-chloro-5-(phenylsulfonyl)-1-(4-piperidinyl)-2,3-dihydro-1H-indole (E19b)

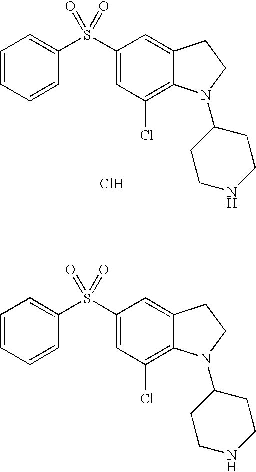

Prepared from 1,1-dimethylethyl 4-[7-chloro-5-(phenylsulfonyl)-2,3-dihydro-1H-indol-1-yl]-1-piperidinecarboxylate (D35) in a similar manner to description (E3a). 7-Chloro-5-(phenylsulfonyl)-1-(4-piperidinyl)-2,3-dihydro-1H-indole hydrochloride (E19a) was crystallised from ethanol and was obtained as a white solid (35%).

¹H-NMR (CDCl₃): δ 1.98 (2H, m), 2.23 (2H, m), 2.98 (4H, m), 3.59 (4H, m), 4.78 (1H, m), 7.39 (1H, d, J=1.6 Hz), 7.47-7.57 (3H, m), 7.62 (1H, d, 1.6 Hz), 7.90 (2H, m), 9.60 (1H, s broad), 9.73 (1H, s broad). MS: m/z (M+H)⁺ 377 & 379, C₁₉H₂₁ClN₂O₂S requires 376 & 378.

The crystallisation residues were passed down an SCX cartridge eluting with methanol and then methanol ammonia. 7-Chloro-5-(phenylsulfonyl)-1-(4-piperidinyl)-2,3-dihydro-1H-indole (E19b) was obtained as a white solid (54%).

¹H-NMR (CDCl₃): δ 1.64 (2H, m), 1.77 (2H, m), 2.68 (2H, td, J=12.2 Hz & 2.0 Hz), 2.96 (2H, t, J=8.8 Hz), 3.14 (2H, m), 3.58 (2H, t, J=9.0 Hz), 4.59 (1H, m), 7.34 (1H, d, J=2.0 Hz), 7.46-7.55 (3H, m), 7.60 (1H, d, J=2.0 Hz), 7.89 (2H, m).

The following examples: E11-E13, E24-E26, were prepared by the coupling of a 1,1-Dimethylethyl 4-(5-iodo-2,3-dihydro-1H-indol-1-yl)-1-piperidinecarboxylate (D6) with sulfonyl fluorides (D18), (D21), (D22), (D49), (D50) and (D51), followed by an acid catalysed deprotection step using methods analogous (see notes column) to those specified in the fully exemplified cases Examples E9 or E10.

TABLE 2

| Example | Sulfonyl | Indolinyl | | | |
|---|---|---|---|---|---|
| number | Fluoride | Halide | Method | Spectral Characterisation | Notes |
| E11 | D22 | D6 | E10 | MS (ES): m/z (M + H)⁺ | |
| | | | | 357; C₂₀H₂₄N₂O₂S | |
| | | | | requires M = 356 | |
| E12 | D49 | D6 | E9 | MS (ES): m/z (M + H)⁺ | Product |
| | | | | 377 and 379; | recrystallised |
| | | | | C₁₉H₂₁ClN₂O₂S requires | from ethanol |
| | | | | M = 376 and 378. | |
| E13 | D51 | D6 | E9 | MS (ES): m/z (M + H)⁺ | Product |
| | | | | 377 and 379; | recrystallised |
| | | | | C₁₉H₂₁ClN₂O₂S requires | from ethanol |
| | | | | M = 376 and 378 | |
| E24 | D50 | D6 | E10 | MS (ES): m/z (M + H)⁺ | Product |
| | | | | 377 and 379; | recrystallised |
| | | | | C₁₉H₂₁ClN₂O₂S requires | from ethanol/ |
| | | | | M = 376 and 378. | diethyl ether |
| E25 | D18 | D6 | E10 | MS (ES): m/z (M + H)⁺ | |
| | | | | 379; C₁₉H₂₀F₂N₂O₂S req. | |
| | | | | M = 378. | |
| E26 | D21 | D6 | E10 | MS (ES): m/z (M + H)⁺ | |
| | | | | 357; C₂₀H₂₄N₂O₂S | |
| | | | | requires M = 356. | |

### EXAMPLE 20

### 2-{[1-(4-Piperidinyl)-2,3-dihydro-1H-indol-5-yl]sulfonyl}-benzonitrile hydrochloride (E20)

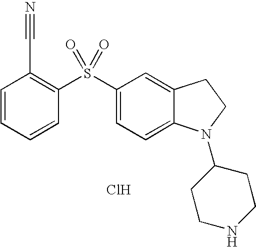

See Table 3

### EXAMPLE 21

### 3-{[1-(4-Piperidinyl)-2,3-dihydro-1H-indol-5-yl]sulfonyl}benzonitrile hydrochloride (E21)

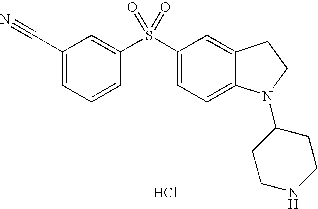

See Table 3

### EXAMPLE 22

### 5-[(3-Fluorophenyl)sulfonyl]-1-(4-piperidinyl)-1H-indole hydrochloride (E22)

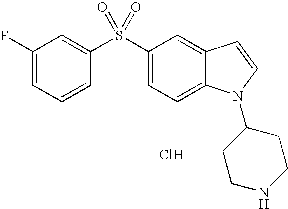

See Table 3

### EXAMPLE 23

### 5-{[3-(Methyloxy)phenyl]sulfonyl}-1-(4-piperidinyl)-2,3-dihydro-1H-indole hydrochloride (E23)

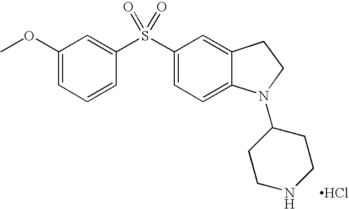

See Table 3

### EXAMPLE 24

### 5-[(3-Chlorophenyl)sulfonyl]-1-(4-piperidinyl)-2,3-dihydro-1H-indole hydrochloride (E24)

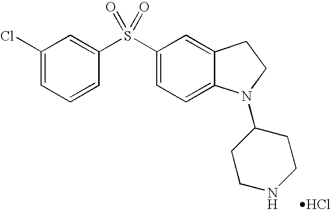

See Table 2

### EXAMPLE 25

### 5-[(3,4-Difluorophenyl)sulfonyl]-1-(4-piperidinyl)-2,3-dihydro-1H-indole hydrochloride (E25)

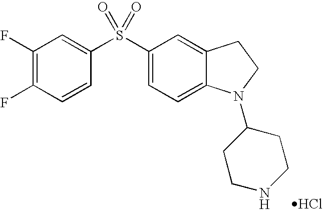

See Table 2

### EXAMPLE 26

### 5-[(3-Methylphenyl)sulfonyl]-1-(4-piperidinyl)-2,3-dihydro-1H-indole hydrochloride (E26)

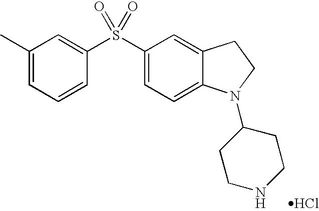

See Table 2

### EXAMPLE 27

### 5-[(2,5-Difluorophenyl)sulfonyl]-1-(4-piperidinyl)-2,3-dihydro-1H-indole hydrochloride (E27)

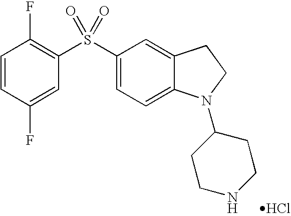

See Table 3

### EXAMPLE 28

### 5-[(2,4-Dimethyl-1,3-thiazol-5-yl)sulfonyl]-1-(4-piperidinyl)-2,3-dihydro-1H-indole hydrochloride (E28)

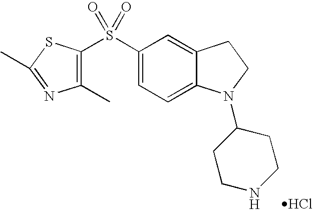

See Table 3

### EXAMPLE 29

### 1-(4-Piperidinyl)-5-[(1,3,5-trimethyl-1H-pyrazol-4-yl)sulfonyl]-2,3-dihydro-1H-indole hydrochloride (E29)

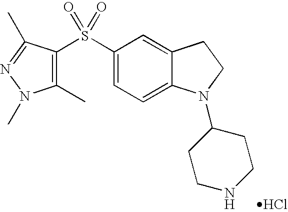

See Table 3

The following examples E7, E14, E20-E23, E27-E29, were prepared by the acid mediated deprotection of the appropriate tert-butyl carbamate D31, D37, D38, D40-D44, D46. In each case the method utilised is analogous (see notes column) to that specified in the fully exemplified cases E1, E3a or E8.

TABLE 3

| Example | Starting | | | |
|---|---|---|---|---|
| number | material | Method | Spectral Characterisation | Notes |
| E7 | D31 | E8 | ¹H NMR (d₆-DMSO) δ 1.22 (3H, | |
| | | | s), 1.83 (2H, m), 2.29 (2H, m), | |
| | | | 2.90 (2H, t J = 8.4 Hz), 3.08 | |
| | | | (4H, m), 3.50 (2H, m, partially | |
| | | | obscured by water), 6.89 (1H, d, | |
| | | | J = 8.4 Hz), 7.50 (2H, m), 7.59 | |
| | | | (3H, m), 7.87 (2H, m) and 8.82 | |
| | | | (1H, br, m). MS (electrospray): | |
| | | | m/z (M + H)⁺ 357; C₂₀H₂₄N₂O₂S | |
| | | | requires M = 356. | |
| E14 | D46 | E1 | MS: m/z (M + H)⁺ 359, | Product precipitated |
| | | | C₁₉H₁₉FN₂O₄S requires 358 | from diethyl ether to |
| | | | | give a white solid |
| E20 | D37 | E3a | MS: m/z (M + H)⁺ 368, | Product crystallised |
| | | | C₂₀H₂₁FN3O₂S req. 367. | from hot ethanol to |
| | | | | give yellow solid |
| E21 | D38 | E3a | MS: m/z (M + H)⁺ 368, | Product purified by |
| | | | C₂₀H₂₁FN₃O₂S req. 367. | HPLC, converted to |
| | | | | HCl salt with 1M |
| | | | | HCl in diethyl ether |
| | | | | and crystallised |
| | | | | from hot ethanol to |
| | | | | give yellow solid |
| E22 | D40 | E1 | MS: m/z (M + H)⁺ 359, | Compound was |
| | | | C₁₉H₁₉FN₂O₄S req. 358. | precipitated from |
| | | | | diethyl ether as |
| | | | | cream solid |
| E23 | D41 | E1 | MS: m/z (M + H⁺)⁺ 373, | Solvent for reaction |
| | | | C₂₀H₂₄N₂O₃S requires 372 | is a mix of |
| | | | | methanol and 4M |
| | | | | HCl in 1,4-dioxane; |
| | | | | reaction time is 1.5 |
| | | | | h |
| E27 | D44 | E1 | MS: m/z (M + H⁺)⁺ 379, | Reaction time is 18 |
| | | | C₁₉H₂₀F₂N₂O₂S requires 378. | hours; product |
| | | | | recrystallised from |
| | | | | iPrOH |
| E28 | D42 | E1 | MS: m/z (M + H⁺)⁺ 378, | Solvent for reaction |
| | | | C₁₈H₂₃N₃O₂S₂ requires 377. | is mix of methanol |
| | | | | and 4M HCl in 1,4- |
| | | | | dioxane; purified by |
| | | | | SCX and Biotage |
| | | | | aminated silica |
| | | | | chromatography; |
| | | | | converted to HCl |
| | | | | salt by treating |
| | | | | methanolic solution |
| | | | | of free base with |
| | | | | 1M HCl in ether |
| E29 | D43 | E1 | MS: m/z (M + H⁺)⁺ 375, | Solvent for reaction |
| | | | C₁₉H₂₆N₄O₂S requires 374. | is 1:2 methanol: |
| | | | | 4M HCl in 1,4- |
| | | | | dioxane; purified |
| | | | | by SCX cartridge |
| | | | | and converted to |
| | | | | HCL salt by treating |
| | | | | methanolic solution |
| | | | | with 1M HCl in |
| | | | | ether |

### EXAMPLE 30

### 1-(1-Methyl-3-pyrrolidinyl)-5-(phenylsulfonyl)-2,3-dihydro-1H-indole hydrochloride (E30)

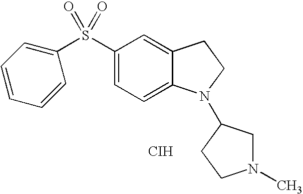

A solution of 5-(phenylsulfonyl)-1-(3-pyrrolidinyl)-2,3-dihydro-1H-indole (E17a) (115 mg, 0.350 mmol, 1.0 equivalents) and formaldehyde (0.13 ml, 37% by wt. in water, 1.75 mmol, 5.0 equivalents) in dichloroethane (2.0 ml) was stirred at room temperature for 10 minutes. Triacetoxyborohydride (297 mg, 1.40 mmol, 4.0 equivalents) was added portionwise and the solution stirred at room temperature for 1.5 hours. The reaction mixture was evaporated to dryness and the residues portioned between dichloromethane (30 ml) and aqueous saturated sodium bicarbonate (20 ml). The organic phase was separated, washed with an additional 15 ml of sodium bicarbonate solution, the brine (15 ml), dried with magnesium sulphate and evaporated to dryness. The resulting colourless oil was purified using an SCX cartridge, eluting with methanol and then methanol ammonia (1M). The appropriate fractions were combined and evaporated to dryness, producing 105 mg of colourless oil. This was then treated with 1M hydrochloric acid in diethyl ether and evaporated to dryness. The product was freeze dried from water. 1-(1-Methyl-3-pyrrolidinyl)-5-(phenylsulfonyl)-2,3-dihydro-1H-indole hydrochloride (E30) was obtained as a white solid (102 mg, 77%).

MS: m/z (M+H)⁺ 343, C₁₉H₂₂N₂O₂S requires 342.

### EXAMPLE 31

### 1-(1-Methylhexahydro-1H-azepin-4-yl)-5-(phenylsulfonyl)-2,3-dihydro-1H-indole hydrochloride (E31)

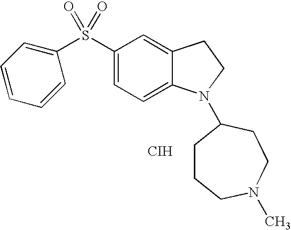

Prepared from 1-(hexahydro-1H-azepin-4-yl)-5-(phenylsulfonyl)-2,3-dihydro-1H-indole (E16a) and formaldehyde in a similar manner to Example 4. The reaction mixture was evaporated to dryness, dissolved in dichloromethane, washed with saturated aqueous sodium bicarbonate twice, then brine, dried with magnesium sulphate, filtered and evaporated to dryness; before purification using an SCX cartridge and treatment with 1M hydrochloric acid in diethyl ether. The product was freeze dried from water. 1-(1-Methylhexahydro-1H-azepin-4-yl)-5-(phenylsulfonyl)-2,3-dihydro-1H-indole hydrochloride (E31) was obtained as a white solid (68%).

MS: m/z (M+H)⁺ 371, C₂₁H₂₆N₂O₂S requires 370.

### EXAMPLE 32

### 7-Chloro-1-(1-methyl-4-piperidinyl)-5-(phenylsulfonyl)-2,3-dihydro-1H-indole hydrochloride (E32)

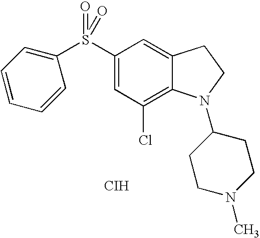

Prepared from 7-chloro-5-(phenylsulfonyl)-1-(4-piperidinyl)-2,3-dihydro-1H-indole (E19b) and formaldehyde in a similar manner to E4. The reaction mixture was evaporated to dryness, dissolved in dichloromethane, washed with saturated aqueous sodium bicarbonate twice, then brine, dried with magnesium sulphate, filtered and evaporated to dryness; before purification using an SCX cartridge and treatment with 1M hydrochloric acid in diethyl ether. 7-Chloro-1-(1-methyl-4-piperidinyl)-5-(phenylsulfonyl)-2,3-dihydro-1H-indole hydrochloride (E32) was crystallised from ethanol with a little diethyl ether and was obtained as a pale yellow solid (60%).

MS: m/z (M+H)⁺ 391 & 393, C₂₀H₂₃ClN₂O₂S requires 390 & 392.

### EXAMPLE 33

### 1-(1-Ethyl-4-piperidinyl)-5-[(3-fluorophenyl)sulfonyl]-1H-indole hydrochloride (E33)

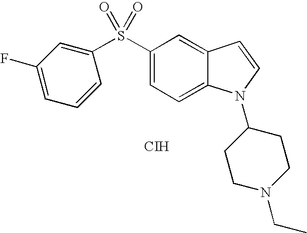

Prepared from 1,1-dimethylethyl 4-{5-[(3-fluorophenyl)sulfonyl]-1H-indol-1-yl}-1-piperidinecarboxylate hydrochloride (E22) in a similar manner to Example 37 but using acetaldehyde in place of 2-methylpropanal. The crude product was purified using Mass Directed Reverse Phase chromatography, an SCX cartridge and then purified on silica, eluting with dichloromethane and methanol (0-20%). The appropriate fractions were combined and evaporated to dryness. The residues were treated with 1M hydrochloric acid in diethyl ether. 1-(1-Ethyl-4-piperidinyl)-5-[(3-fluorophenyl)sulfonyl]-1H-indole hydrochloride (E33) was precipitated from diethyl ether and was obtained as a yellow solid (3%).

MS: m/z (M+H)⁺ 387, C₂₁H₂₃FN₂O₂S requires 386.

### EXAMPLE 34

### 5-[(3,5-Difluorophenyl)sulfonyl]-1-(1-propyl-4-piperidinyl)-2,3-dihydro-1H-indole hydrochloride (E34)

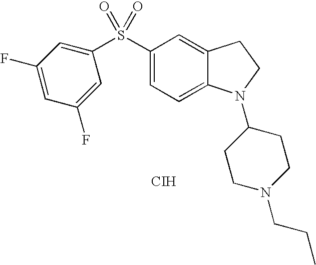

Prepared from 5-[(3,5-difluorophenyl)sulfonyl]-1-(4-piperidinyl)-2,3-dihydro-1H-indole hydrochloride (E10) and propionaldehyde in a similar manner to Example 37 however, the product was not purified by Mass Directed Reverse Phase chromatography, but instead was purified on silica eluting with dichloromethane and methanol (0-20%). The appropriate fractions were combined and evaporated to dryness. The residues were dissolved in dichloromethane and treated with 1M hydrochloric acid in diethyl ether. 5-[(3,5-Difluorophenyl)sulfonyl]-1-(1-propyl-4-piperidinyl)-2,3-dihydro-1H-indole hydrochloride (E34) was then crystallised from propan-2-ol, and was obtained as a yellow solid (47%).

MS: m/z (M+H)⁺ 421, C₂₂H₂₆F₂N₂O₂S requires 420.

### EXAMPLE 35

### 1-(1-Ethyl-4-piperidinyl)-5-[(3,5-difluorophenyl)sulfonyl]-1H-indole hydrochloride (E35)

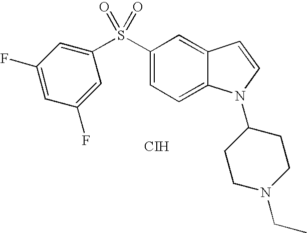

This was prepared from 5-[(3,5-difluorophenyl)sulfonyl]-1-(1-ethyl-4-piperidinyl)-2,3-dihydro-1H-indole hydrochloride (E51) using 2,3-dichloro-5,6-dicyano-1,4-benzoquinone using a method analogous to that described in D2. The crude product was purified on silica, eluting with dichloromethane and methanol (0-20%), and then using Mass Directed Reverse Phase chromatography. The product was dissolved in dichloromethane and treated with 1M hydrochloric acid in diethyl ether. 1-(1-Ethyl-4-piperidinyl)-5-[(3,5-difluorophenyl)sulfonyl]-1H-indole hydrochloride (E35) was crystallised from hot propan-2-ol and was obtained as a white solid (5%).

MS: m/z (M+H)⁺ 405, C₂₁H₂₂F₂N₂O₂S requires 404.

### EXAMPLE 36

### 1-[1-(1-Methylethyl)-4-piperidinyl]-5-(phenylsulfonyl)-2,3-dihydro-1H-indole

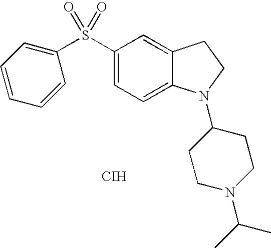

A suspension of 5-(phenylsulfonyl)-1-(4-piperidinyl)-2,3-dihydro-1H-indole (E3b) (150 mg, 0.396 mmol, 1.0 equivalents), sodium triacetoxyborohydride (336 mg, 1.58 mmol, 4.0 equivalents) and acetone (0.044 ml, 0.594 mmol, 1.5 equivalents) in dichloroethane (3 ml) was stirred at room temperature for 40 h. The crude product was purified using an SCX cartridge, eluting with methanol and then methanol/ammonia. The appropriate fractions were evaporated to dryness. The residues were partitioned between ethyl acetate and water. The organic phase was separated, washed with water, dried with magnesium sulphate, filtered, and evaporated to dryness. The residues were treated with 1M hydrochloric acid in diethyl ether, evaporated to dryness and precipitated from diethyl ether. 1-[1-(2-Methylethyl)-4-piperidinyl]-5-(phenylsulfonyl)-2,3-dihydro-1H-indole hydrochloride (E36) was obtained as a white solid (26%).

¹H-NMR (CDCl₃): δ 1.46 (6H, m), 1.94 (2H, m), 2.84 (4H, m), 3.00 (2H, t, J=8.2 Hz), 3.51 (3H, m), 3.61-3.74 (3H, m), 6.31 (1H, m), 7.44-7.53 (4H, m), 7.67 (1H, m), 7.89 (2H, d, J=6.8 Hz), 12.3 (1H, s broad).

MS: m/z (M+H)⁺ 385, C₂₂H₂₈N₂O₂S requires 384.

### EXAMPLE 37

### 1-[1-(2-Methylpropyl)-4-piperidinyl]-5-(phenylsulfonyl)-2,3-dihydro-1H-indole hydrochloride (E37)

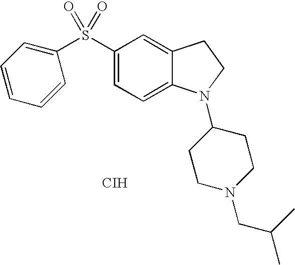

A suspension of 5-(phenylsulfonyl)-1-(4-piperidinyl)-2,3-dihydro-1H-indole (E3b) (100 mg, 0.291 mmol, 1.0 equivalents), sodium triacetoxyborohydride (93 mg, 0.438 mmol, 1.5 equivalents) and 2-methylpropanal (0.040 ml, 0.438 mmol, 1.5 equivalents) in 1,2-dichloroethane (2 ml) was stirred at room temperature for 20 h. The reaction mixture was partitioned between saturated aqueous sodium bicarbonate (20 ml) and dichloromethane (20 ml). The organic phase was separated, washed with brine (20 ml), dried with magnesium sulphate, filtered, and evaporated to dryness. The reaction mixture was purified by Mass Directed Reverse Phase chromatography, producing the formate salt of the product, which was then dissolved in dichloromethane and treated with 1M hydrochloric acid in diethyl ether. 1-[1-(2-methylpropyl)-4-piperidinyl]-5-(phenylsulfonyl)-2,3-dihydro-1H-indole hydrochloride (E37) was then precipitation from diethyl ether, and was obtained as a white solid (61 mg, 48%).

¹H-NMR (CDCl₃): δ 0.91 (2H, m), 1.18 (2H, d), 1.33 (2H, m), 1.89 (2H, m), 2.24 (1H, m) 2.80-2.92 (4H, m), 3.01 (2H, t), 3.61-3.70 (4H, m), 4.22 (1H, m), 6.32 (1H, m), 7.45-7.56 (4H, m), 7.68 (1H, m), 7.71 (2H, m), 12.2 (1H, s broad). MS: m/z (M+H)⁺ 399, C₂₃H₃₀N₂O₂S requires 398.

### EXAMPLE 38

### 1-[1-(2,2-dimethylpropyl)-4-piperidinyl]-5-(phenylsulfonyl)-2,3-dihydro 1H-indole (E38)

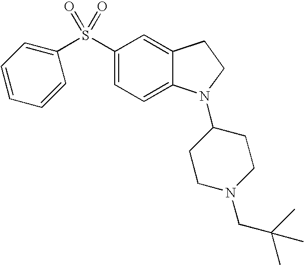

See Table 4.

### EXAMPLE 39

### 1-(1-Ethyl-4-piperidinyl)-5-(phenylsulfonyl)-2,3-dihydro-1H-indole hydrochloride (E39)

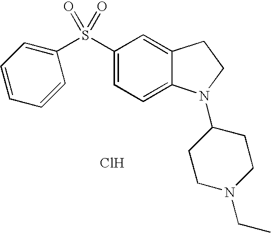

See Table 4.

### EXAMPLE 40

### 5-(Phenylsulfonyl)-1-(1-propyl-4-piperidinyl)-2,3-dihydro-1H-indole hydrochloride (E40)

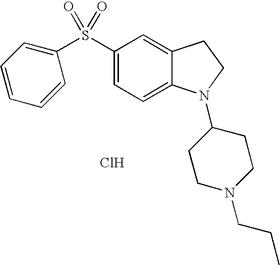

See Table 4.

### EXAMPLE 41

### 5-[(4-Fluorophenyl)sulfonyl]-1-(1-methyl-4-piperidinyl)-2,3-dihydro-1H-indole hydrochloride (E41)

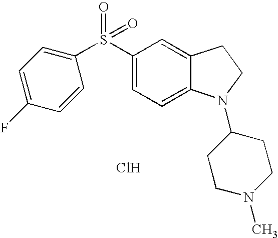

See Table 4.

### EXAMPLE 42

### 5-[(2,5-Difluorophenyl)sulfonyl]-1-(1-ethyl-4-piperidinyl)-2,3-dihydro-1H-indole hydrochloride (E42)

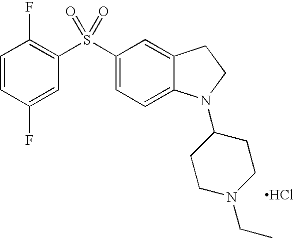

See Table 4.

### EXAMPLE 43

### 5-[(2,5-Difluorophenyl)sulfonyl]-1-[1-(1-methylethyl)-4-piperidinyl]-2,3-dihydro-1H-indole hydrochloride (E43)

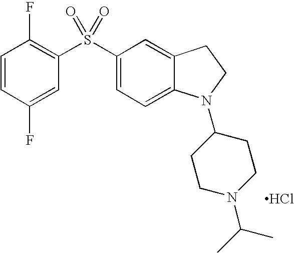

See Table 4.

### EXAMPLE 44

### 5-[(2,5-Difluorophenyl)sulfonyl]-1-(1-propyl-4-piperidinyl)-2,3-dihydro-1H-indole hydrochloride (E44)

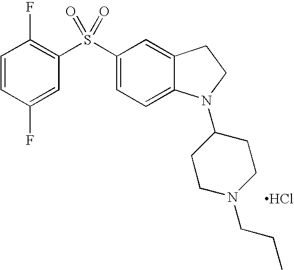

See Table 4.

### EXAMPLE 45

### 5-[(2-Fluorophenyl)sulfonyl]-1-[1-(1-methylethyl)-4-piperidinyl]-2,3-dihydro-1H-indole hydrochloride (E45)

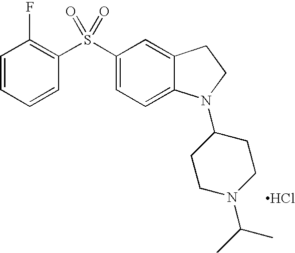

See Table 4.

### EXAMPLE 46

### 5-[(2-Fluorophenyl)sulfonyl]-1-(1-propyl-4-piperidinyl)-2,3-dihydro-1H-indole hydrochloride (E46)

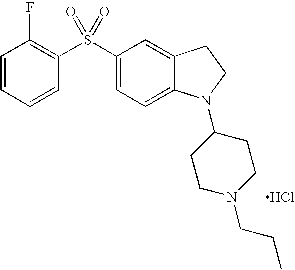

5-[(2-Fluorophenyl)sulfonyl]-1-(4-piperidinyl)-2,3-dihydro-1H-indole hydrochloride (GSK703427A) (E9) (370 mg, 0.928 mmol) was dissolved in 1,2-dichloroethane (11 ml) and propionaldehyde (270 mg, 4.64 mmol) was added. After 10 minutes NaHB(OAc)₃ (787 mg, 3.71 mmol) was added and the mixture was left stirring for 18 hrs. The reaction mixture was diluted with dichloromethane (40 ml), washed with potassium carbonate (5%, 2×25 ml), brine and dried over MgSO₄. The solution was concentrated to afford the crude material as a yellow oil (322 mg) which was purified by flash chromatography (Flashmaster, 20 g cartridge) with a gradient of MeOH (0-5%) in dichloromethane. The desired product as free base (281 mg, 0.7 mmol) was dissolved in a small amount of MeOH and treated with HCl (1M in Et₂O, 0.77 mmol, 0.77 ml) to make the HCl salt; the solvent was removed and resulting white solid was triturated with hexane first and then recrystallised from isopropanol (230 mg in ca. 20 ml). the desired product 5-[(2-fluorophenyl)sulfonyl]-1-(1-propyl-4-piperidinyl)-2,3-dihydro-1H-indole hydrochloride (E46) was isolated as white crystals (183 mg, 45%).

¹H-NMR ((CD₃)₂SO): δ 0.91 (3H, t), 1.70 (2H, m), 1.84 (2H, d), 2.05 (2H, q), 2.99 (6H, m), 3.52 (4H, t), 3.87 (1H, m), 6.62 (1H, d), 7.41 (3H, m), 7.60 (1H, d), 7.70 (1H, m), 7.96 (1H, t).

MS: m/z (M+H⁺)+403, C₂₂H₂₇FN₂O₂S requires 402.

### EXAMPLE 47

### 5-[(3-Fluorophenyl)sulfonyl]-1-(1-methyl-4-piperidinyl)-2,3-dihydro-1H-indole hydrochloride (E47)

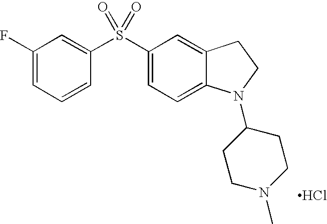

A suspension of 5-[(3-fluorophenyl)sulfonyl]-1-(4-piperidinyl)-2,3-dihydro-1H-indole hydrochloride (E8) (111 mg, 0.28 mmol) in 1,2-dichloroethane (3 ml) was treated with 37% aqueous formaldehyde (0.113 ml, 1.4 mmol) and the mixture was stirred at RT for 10 minutes. Sodium triacetoxyborohydride (237 mg, 1.12 mmol) was then added and the mixture was stirred at RT for an additional 2 h. Dichloromethane (20 ml) was then added and the resulting mixture was washed with 5% aqueous K₂CO₃ solution (2×10 ml) and brine (1×10 ml). The organic solution was dried over MgSO₄, filtered and evaporated to leave a pale yellow oil. This was dissolved in dichloromethane (3 ml) and treated with 1M HCl in diethyl ether. The resulting cloudy solution was evaporated to dryness and the residue was triturated with diethyl ether to afford a white solid. This was filtered and dried in vacuo to afford 5-[(3-fluorophenyl)sulfonyl]-1-(1-methyl-4-piperidinyl)-2,3-dihydro-1H-indole hydrochloride (E47), 63 mg (55%).

MS (electrospray): m/z (M+H)⁺ 375; C₂₀H₂₃FN₂O₂S requires M=374.

### EXAMPLE 48

### 5-[(2-Fluorophenyl)sulfonyl]-1-(1-methyl-4-piperidinyl)-2,3-dihydro-1H-indole hydrochloride (E48)

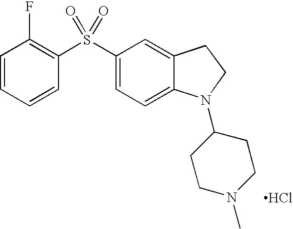

See Table 4.

### EXAMPLE 49

### 5-[(2-Chlorophenyl)sulfonyl]-1-(1-methyl-4-piperidinyl)-2,3-dihydro-1H-indole hydrochloride (E49)

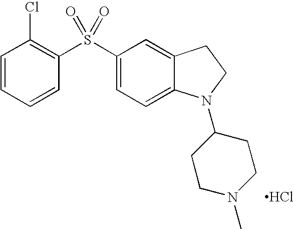

See Table 4.

### EXAMPLE 50

### 5-[(3,5-Difluorophenyl)sulfonyl]-1-(1-methyl-4-piperidinyl)-2,3-dihydro-1H-indole hydrochloride (E50)

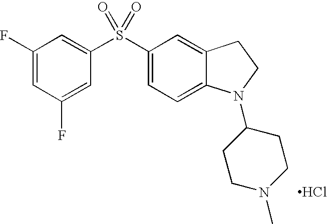

See Table 4.

### EXAMPLE 51

### 5-[(3,5-Difluorophenyl)sulfonyl]-1-(1-ethyl-4-piperidinyl)-2,3-dihydro-1H-indole hydrochloride (E51)

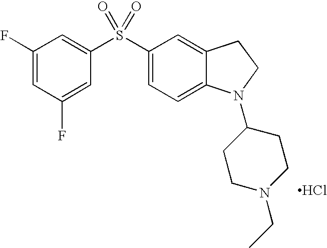

See Table 4.

### EXAMPLE 52

### 5-[(3,5-Difluorophenyl)sulfonyl]-1-[1-(1-methylethyl)-4-piperidinyl)-2,3-dihydro-1H-indole hydrochloride (E52)

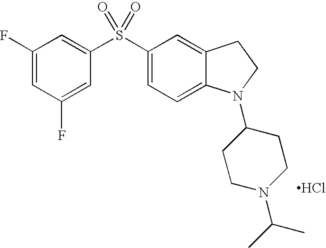

See Table 4.

### EXAMPLE 53

### 5-[(3-Chlorophenyl)sulfonyl]-1-(1-methyl-4-piperidinyl)-2,3-dihydro-1H-indole hydrochloride (E53)

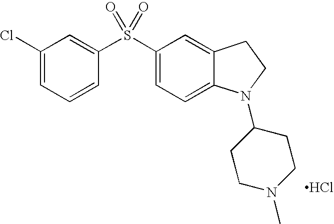

See Table 4.

### EXAMPLE 54

### 5-[((3-Chlorophenyl)sulfonyl]-1-(1-ethyl-4-piperidinyl)-2,3-dihydro-1H-indole hydrochloride (E54)

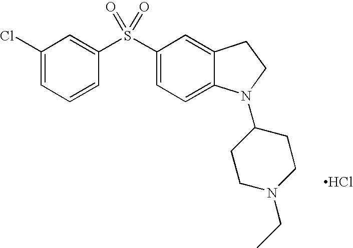

See Table 4.

### EXAMPLE 55

### 5-[(3-Fluorophenyl)sulfonyl]-1-(1-ethyl-4-piperidinyl)-2,3-dihydro-1H-indole hydrochloride (E55)

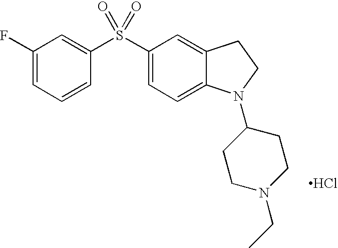

See Table 4.

### EXAMPLE 56

### 5-[(3-Fluorophenyl)sulfonyl]-1-(1-propyl-4-piperidinyl)-2,3-dihydro-1H-indole hydrochloride (E56)

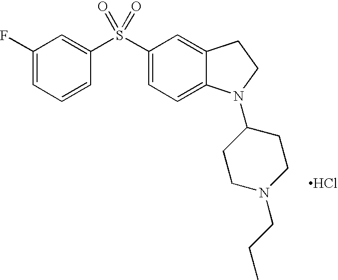

See Table 4.

### EXAMPLE 57

### 5-[(3-Fluorophenyl)sulfonyl]-1-(1-(1-methylethyl)-4-piperidinyl)-2,3-dihydro-1H-indole hydrochloride (E57)

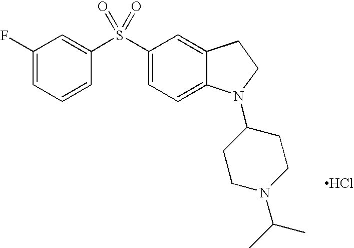

See Table 4.

### EXAMPLE 58

### 5-[(2-Fluorophenyl)sulfonyl]-1-(1-ethyl-4-piperidinyl)-2,3-dihydro-1H-indole hydrochloride (E58)

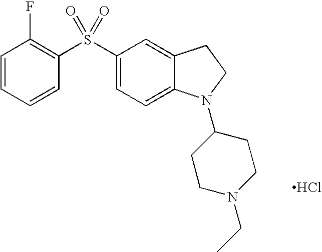

See Table 4.

### EXAMPLE 59

### 5-[(3,4-Difluorophenyl)sulfonyl]-1-(1-methyl-4-piperidinyl)-2,3-dihydro-1H-indole hydrochloride (E59)

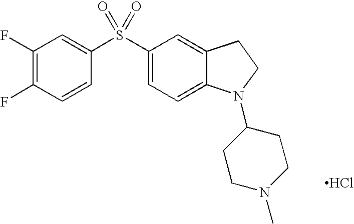

See Table 4.

### EXAMPLE 60

### 5-[(3,4-Difluorophenyl)sulfonyl]-1-(1-ethyl-4-piperidinyl)-2,3-dihydro-1H-indole hydrochloride (E60)

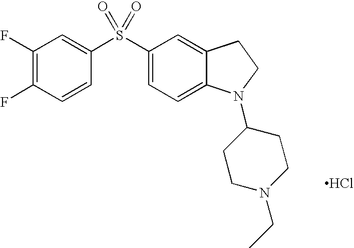

See Table 4.

### EXAMPLE 61

### 5-[(3,4-Difluorophenyl)sulfonyl]-1-(1-propyl-4-piperidinyl)-2,3-dihydro-1H-indole hydrochloride (E61)

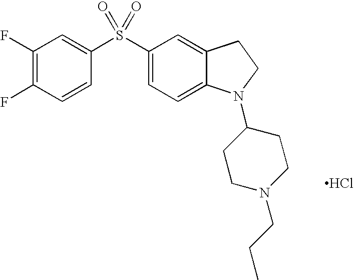

See Table 4.

### EXAMPLE 62

### 5-[(3,4-Difluorophenyl)sulfonyl]-1-(1-(1-methylethyl)-4-piperidinyl)-2,3-dihydro-1H-indole hydrochloride (E62)

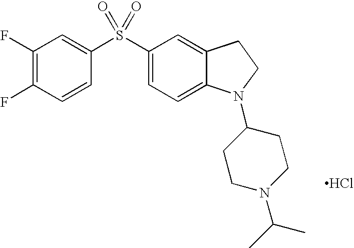

See Table 4.

### EXAMPLE 63

### 5-[(3-Methylphenyl)sulfonyl]-1-(1-ethyl-4-piperidinyl)-2,3-dihydro-1H-indole hydrochloride (E63)

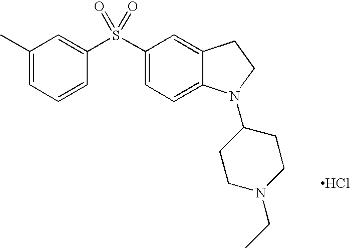

See Table 4.

### EXAMPLE 64

### 5-[(3-Methylphenyl)sulfonyl]-1-(1-propyl-4-piperidinyl)-2,3-dihydro-1H-indole hydrochloride (E64)

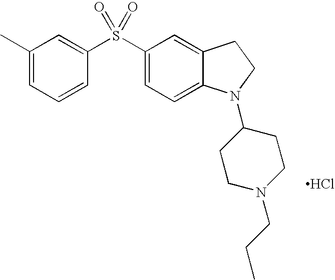

See Table 4.

The following examples: E38-E45, E48-E64 were prepared by the reductive amination of secondary amine examples: E8, E9, E10, E12, E24, E25, E26, E27, using the specified carbonyl compounds. In each case the method utilised is analogous (see notes column) to that specified in the fully exemplified cases: E30, E37, E46 or E47.

TABLE 4

| Example | Starting | Carbonyl | | Spectral | |
|---|---|---|---|---|---|
| number | amine | Compound | Method | characterisation | Notes |
| E38 | E3b | 2,2-dimethy- | E37 | MS: m/z (M + H) + | Free base - white |
| | | Ipropanal | | 413, C24H32N2O2S | solid crystallised |
| | | | | requires 412. | from methanol - |
| | | | | | not purified |
| | | | | | further or |
| | | | | | converted to salt |
| E39 | E3b | acetaldehyde | E37 | MS: m/z (M + H) + | White solid |
| | | | | 371, C21H26N2O2S | |
| | | | | requires 370 | |
| E40 | E3b | Propion- | E37 | MS: m/z (M + H) + | Cream solid |
| | | aldehyde | | 385, C22H28N2O2S | |
| | | | | requires 384. | |
| E41 | E15b | Formalin | E30 | MS: m/z (M + H) + | Crystallised from |
| | | | | 375, | propanon-2-ol |
| | | | | C20H23FN2O2S | and diethyl ether |
| | | | | requires 374 | as a |
| | | | | | white solid |
| E42 | E27 | Acetaldehyde | E46 | MS: m/z (M + H+) + | Salt isolated |
| | | | | 407, | without |
| | | | | C21H24F2N2O2S | crystallisation or |
| | | | | requires 406 | trituration |
| E43 | E27 | Acetone | E46 | MS: m/z (M + H+) + | Salt isolated |
| | | | | 421, | without |
| | | | | C22H26F2N2O2S | crystallisation or |
| | | | | requires 420 | trituration |
| E44 | E27 | Propion- | E46 | MS: m/z (M + H+) + | Salt isolated |
| | | aldehyde | | 421, | without |
| | | | | C22H26F2N2O2S | crystallisation or |
| | | | | requires 420 | trituration |
| E45 | E9 | Acetone | E46 | MS: m/z (M + H+) + | Reaction time = 5 |
| | | | | 403, | days |
| | | | | C22H27FN2O2S | |
| | | | | requires 402. | |
| E48 | E9 | Formalin | E47 | MS (ES): m/z | |
| | | | | (M + H) + 375; | |
| | | | | C20H23FN2O2S | |
| | | | | requires M = 374 | |
| E49 | E12 | Formalin | E47 | MS (ES): m/z | Reaction time = |
| | | | | (M + H) + 391 and | 3 h; product |
| | | | | 393; | recryst. From |
| | | | | C20H23ClN2O2S | EtOH |
| | | | | requires M = 390 | |
| | | | | and 392. | |
| E50 | E10 | Formalin | E47 | MS (ES): m/z | Reaction time = |
| | | | | (M + H) + 393; | 3.5 h |
| | | | | C20H22F2N2O2S | |
| | | | | requires M = 392 | |
| E51 | E10 | Acetaldehyde | E47 | MS (ES): m/z | Reaction time = |
| | | | | (M + H) + 407; | 20 h |
| | | | | C21H24F2N2O2S | |
| | | | | requires M = 406 | |
| E52 | E10 | Acetone | E47 | MS (ES): m/z | Reaction time = 2 |
| | | | | (M + H) + 421; | days |
| | | | | C22H26F2N2O2S | |
| | | | | req. M = 420. | |
| E53 | E24 | Formalin | E47 | MS (ES): m/z | Reaction time = |
| | | | | (M + H) + 391 and | 14 h |
| | | | | 393; | |
| | | | | C20H23ClN2O2S | |
| | | | | req. M = 390 and | |
| | | | | 392. | |
| E54 | E24 | Acetaldehyde | E47 | MS (ES): m/z | Reaction time = |
| | | | | (M + H) + 405 and | 14 h; product |
| | | | | 407; | purified by HPLC |
| | | | | C21H25ClN2O2S | before conversion |
| | | | | req. M = 404 and | to HCl salt as in |
| | | | | 406 | E54 |
| E55 | E8 | Acetaldehyde | E47 | MS (ES): m/z | Reaction time = |
| | | | | (M + H) + 389; | 14 h; product |
| | | | | C21H25FN2O2S | recryst. From |
| | | | | requires M = 388 | iPrOH |
| E56 | E8 | Propion- | E47 | MS (ES): m/z | Reaction time = |
| | | aldehyde | | (M + H) + 403; | 14 h; product |
| | | | | C22H27FN2O2S | recryst. From |
| | | | | requires M = 402 | iPrOH/EtOH |
| E57 | E8 | Acetone | E47 | MS (ES): m/z | Reaction time = 5 |
| | | | | (M + H) + 403; | days; product |
| | | | | C22H27FN2O2S | purified by HPLC |
| | | | | req. M = 402 | before conversion |
| | | | | | to HCl salt as in |
| | | | | | E54 |
| E58 | E9 | Acetaldehyde | E47 | MS (ES): m/z | |
| | | | | (M + H) + 389; | |
| | | | | C21H25FN2O2S | |
| | | | | req. M = 388 | |
| E59 | E25 | Formalin | E47 | MS (ES): m/z | Reaction time = 3 |
| | | | | (M + H) + 393; | days; product |
| | | | | C20H22F2N2O2S | purified by |
| | | | | req. M = 392. | chromatography |
| | | | | | on silica gel |
| | | | | | eluting with 0-3% |
| | | | | | MeOH in CH₂Cl₂ |
| | | | | | prior to HCl salt |
| | | | | | formation |
| E60 | E25 | Acetaldehyde | E47 | MS (electrospray): | Reaction time = 3 |
| | | | | m/z (M + H) + 407; | days product |
| | | | | C21H24F2N2O2S | purified by |
| | | | | requires M = 406. | chromatography |
| | | | | | on silica gel |
| | | | | | eluting with 0-3% |
| | | | | | MeOH in CH₂Cl₂ |
| | | | | | prior to HCl salt |
| | | | | | formation |
| E61 | E25 | Propion- | E47 | MS (ES): m/z | Reaction time = 3 |
| | | aldehyde | | (M + H) + 421; | days product |
| | | | | C22H26F2N2O2S | purified by |
| | | | | req. M = 420. | chromatography |
| | | | | | on silica gel |
| | | | | | eluting with 0-3% |
| | | | | | MeOH in CH₂Cl₂ |
| | | | | | prior to HCl salt |
| | | | | | formation |
| E62 | E25 | Acetone | E47 | MS (ES): m/z | Reaction time = 6 |
| | | | | (M + H) + 421; | days product |
| | | | | C22H26F2N2O2S | purified by |
| | | | | req. M = 420. | chromatography |
| | | | | | on silica gel |
| | | | | | eluting with 0-3% |
| | | | | | MeOH in CH₂Cl₂ |
| | | | | | prior to HCl salt |
| | | | | | formation |
| E63 | E26 | Acetaldehyde | E47 | MS (ES): m/z | Reaction time = |
| | | | | (M + H) + 385; | 1.5 h product |
| | | | | C22H28N2O2S req. | purified by |
| | | | | M = 384. | chromatography |
| | | | | | on silica gel |
| | | | | | eluting with 0- |
| | | | | | 7.5% MeOH in |
| | | | | | CH₂Cl₂ prior to |
| | | | | | HCl salt formation |
| E64 | E26 | Propion- | E47 | MS (ES): m/z | Reaction time = |
| | | aldehyde | | (M + H) + 399; | 1.5 h product |
| | | | | C23H30N2O2S req. | purified by |
| | | | | M = 398. | chromatography |
| | | | | | on silica gel |
| | | | | | eluting with 0- |
| | | | | | 7.5% MeOH in |
| | | | | | CH₂Cl₂ prior to |
| | | | | | HCl salt formation |

### EXAMPLE 65

### 3-Methyl-5-(phenylsulfonyl)-1-(4-piperidinyl)-1H-indole hydrochloride (E65)

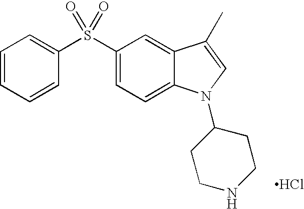

1,1-Dimethylethyl 4-[3-methyl-5-(phenylsulfonyl)-1H-indol-1-yl]-1-piperidinecarboxylate (D54) (80 mg, 0.175 mmol) was treated with 4M HCl in dioxane (5 ml) as described in Example E8 to afford 3-methyl-5-(phenylsulfonyl)-1-(4-piperidinyl)-1H-indole hydrochloride (E65) as a white solid (54 mg, 95%).

¹H NMR (d₆-DMSO) δ 2.05 (2H, m), 2.16 (2H, m), 2.32 (3H, s), 3.10 (2H, m), 3.40 (2H, m, obscured by H₂O), 4.75 (1H, m), 7.38 (1H, s), 7.62 (3H, m), 7.68 (1H, m), 7.76 (1H, d J=8.8 Hz), 7.96 (2H, m), 8.17 (1H, s) and 8.89 (2H, br. d). MS (electrospray): m/z (M+H)⁺ 354; C₂₀H₂₂N₂O₂S requires M=354.

### Pharmacological Data

Compounds of the invention may be tested for in vitro biological activity in accordance with the following cyclase assay:

### Cyclase Assay

0.5 μl of test compound in 100% dimethylsulfoxide (DMSO) was added to a white, solid 384 well assay plate (for dose response measurements the top of the concentration range is 7.5 μM final). 10 μl of washed membranes of HeLa 5HT₆ cells (for preparation see WO 98/27081) in basic buffer (50 mM HEPES pH 7.4 (KOH), 10 mM MgCl₂, 100 mM NaCl, 10 μM 3-isobutyl-1-methylxanthine (IBMX) (Sigma-Aldrich)) was added to all wells followed by 10 μl 2×ATP buffer (i.e. basic buffer containing 3 mM ATP) with 5-HT (at a concentration equivalent to a dose response of 4×EC₅₀). The resultant mixture was then incubated at room temperature for 30-45 minutes to allow cAMP production. cAMP production was then measured using the DiscoveRX™ HitHunter™ chemiluminescence cAMP assay kit (DiscoveRx Corporation, 42501 Albrae Street, Fremont, Calif. 94538; Product Code: 90-0004L) or any other suitable cAMP measurement assay.

IC₅₀ values were estimated from arbitrary designated unit (ADU) measurements from a Perkin Elmer Viewlux instrument using a four parameter logistic curve fit within EXCEL (Bowen, W. P. and Jerman, J. C. (1995), Nonlinear regression using spreadsheets. Trends in Pharmacol. Sci., 16, 413-417). Functional Kᵢ values were calculated using the method of Cheng, Y. C. and Prussof, W. H. (Biochemical Pharmacol (1973) 22 3099-3108). plC₅₀ and fpKᵢ are the negative log 10 of the molar IC₅₀ and functional Kᵢ respectively.

The compounds of Examples E1-4, 6, 8-28, 30-37, 39-60 and 63-65 were tested in the above cyclase assay and showed affinity for the 5-HT₆ receptor, having pKᵢ values ≧8.0 at human cloned 5-HT₆ receptors. The compounds of Examples E5, 7, 29, 38 and 61-62 were also tested in the above cyclase assay and showed affinity for the 5-HT₆ receptor, having pKᵢ values >6.5 at human cloned 5-HT₆ receptors.

1. A compound of formula (I) or a pharmaceutically acceptable salt or solvate thereof: 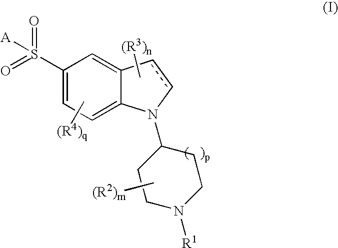
wherein:
R¹ represents hydrogen or C₁₋₆ alkyl optionally substituted by one or more halogen or cyano groups;
R² represents C₁₋₆ alkyl or R² may be linked to R¹ to form a (CH₂)₂, (CH₂)₃ or (CH₂)₄ group;
m represents an integer from zero to 4, such that when m is greater than 1, two R² groups may be linked to form a CH₂, (CH₂)₂, CH₂OCH₂ or (CH₂)₃ group;
p represents an integer from zero to 2; [symbol]
represents a single or a double bond;
R³ represents C₁₋₆ alkyl or ═O;
n represents an integer from zero to 2;
R⁴ represents halogen, cyano, haloC₁₋₆ alkyl, haloC₁₋₆ alkoxy, C₁₋₆ alkyl, C₁₋₆ alkoxy, C₁₋₆ alkanoyl or a group —CONR⁵R⁶;
q represents an integer from zero to 3;
R⁵ and R⁶ independently represent hydrogen or C₁₋₆ alkyl or together with the nitrogen atom to which they are attached form a nitrogen containing heterocyclyl or nitrogen containing heteroaryl group;
A represents an -aryl, -heteroaryl, -aryl-aryl, -aryl-heteroaryl, -heteroaryl-aryl or -heteroaryl-heteroaryl group;
wherein said aryl and heteroaryl groups of A may be optionally substituted by one or more substituents which may be the same or different, and which are selected from the group consisting of halogen, hydroxy, cyano, nitro, trifluoromethyl, trifluoromethoxy, C₁₋₆ alkyl, trifluoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₆ alkyl, C₁₋₆ alkylsulfonamido, C₁₋₆ alkylamido, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, CONR⁹R¹⁰ and SO₂NR⁹R¹⁰, wherein R⁹ and R¹⁰ independently represent hydrogen or C₁₋₆ alkyl or R⁹ and R¹⁰ together with the nitrogen atom to which they are attached may form a nitrogen containing heterocyclyl or nitrogen containing heteroaryl group.

2. A compound of formula (I) as defined in claim 1 or a pharmaceutically acceptable salt or solvate thereof, wherein R¹ represents hydrogen or C₁₋₆ alkyl.

3. A compound of formula (I) as defined in claim 1 or a pharmaceutically acceptable salt or solvate thereof, wherein A represents an optionally substituted phenyl, thiazolyl or pyrazolyl, wherein the optional substituents are selected from the group consisting of halogen, CN, C₁₋₃ alkyl and C₁₋₃ alkoxy.

4. A compound of formula (I) as defined in claim 1 or a pharmaceutically acceptable salt or solvate thereof, which is a compound of E1-E65.

5. A pharmaceutical composition which comprises a compound as defined in claim 1, or a pharmaceutically acceptable salt or solvate thereof, and a pharmaceutically acceptable carrier or excipient.

6-9. (canceled)

10. A method of treating depression, anxiety, Alzheimer's disease, age related cognitive decline, ADHD, obesity, mild cognitive impairment, schizophrenia, cognitive deficits in schizophrenia and stroke which comprises administering a safe and therapeutically effective amount to a patient in need thereof of a compound of formula (I) as defined in claim 1 or a pharmaceutically acceptable salt or solvate thereof.
